(19) United States
(12) Patent Application Publication
(10) Pub. No.: US 2022/0143142 A1
(43) Pub. Date: May 12, 2022

(54) DIRECT REPROGRAMMING OF CARDIAC FIBROBLASTS INTO CARDIOMYOCYTES USING AN ENDOTHELIAL CELL TRANSDIFFERENTIATION STRATEGY

(71) Applicant: Baylor College of Medicine, Houston, TX (US)

(72) Inventors: Megumi Mathison, Houston, TX (US); Todd Rosengart, Houston, TX (US); Vivek P. Singh, Houston, TX (US); Deepthi Sanagasetti, Pearland, TX (US); Jaya Pratap Pinnamaneni, Houston, TX (US); Jianchang Yang, Pearland, TX (US)

(21) Appl. No.: 17/593,286

(22) PCT Filed: Mar. 17, 2020
(86) PCT No.: PCT/US2020/023145

§ 371 Date: Sep. 14, 2021

Related U.S. Application Data
(60) Provisional application No. 62/830,543, filed on Apr. 7, 2019.
(60) Provisional application No. 62/819,636, filed on Mar. 17, 2019.

(51) Int. Cl. A61K 38/18; A61K 38/17; A61K 35/34; A61K 45/06; C12N 5/077

(52) U.S. Cl. CPC A61K 38/1866; A61K 38/1709; A61K 35/34; C12N 2501/90; C12N 5/0656; C12N 2506/28; C12N 2501/998; A61K 45/06

(57) ABSTRACT

Embodiments of the disclosure provide methods and compositions related to improving cardiomyocyte production by exposing starting cells to ETV2 and/or VEGF. The starting cells in specific embodiments are fibroblasts and/or endothelial cells, and following exposure to ETV2 and/or VEGF the resultant cells are exposed to one or more cardiomyocyte transdifferentiation factors, such as GATA4, myocyte enhancer factor-2c (Mef2c), T-box transcription factor 5 (TBX5), or a combination thereof. The produced cardiomyocytes are provided to individuals in need thereof, in particular embodiments.

### CROSS REFERENCE TO RELATED APPLICATIONS

This application claims the benefit of priority to U.S. Provisional Patent Application Ser. Nos. 62/819,636 and 62/830,543, filed Mar. 17, 2019, and Apr. 7, 2019, hereby incorporated by reference in their entirety.

### STATEMENT REGARDING FEDERALLY SPONSORED RESEARCH OR DEVELOPMENT

This invention was made with government support under HL121294 awarded by National Institutes of Health. The government has certain rights in the invention.

### TECHNICAL FIELD

Embodiments of the disclosure include at least the fields of cell biology, molecular biology, physiology, biology, and medicine, including cardiac medicine.

### BACKGROUND

Since the possibility of cardiac cellular reprogramming was reported in 2010, a wide variety of reprogramming cocktails have been utilized to induce the transdifferentiation of cardiac fibroblasts into “induced cardiomyocytes” (iCMs) and thereby improve post-infarct cardiac function in small animal models. Limits on cardiac transdifferentiation efficiency that are exaggerated in human cells and other higher order species have catalyzed the search for alternative paradigms for effective cardiac reprogramming strategies that might be translatable to human applications. Enhancing the plasticity—or the susceptibility of cells to reprogramming—has been a major theme of these strategies.

The present disclosure satisfies a long felt need in the art of effectively producing cardiomyocytes for therapeutic applications.

### BRIEF SUMMARY

Embodiments of the disclosure concern methods and compositions related to cardiac medicine, including improvements on existing methods and compositions for cardiac medicine. In particular embodiments, the disclosure provides methods and compositions for cardiac tissue repair and regeneration by generating cardiomyocytes for individuals in need thereof. The cardiomyocytes may be used to improve cardiac function, particular in cases wherein there has been tissue damage, such as in a post-infarct individual, as one example.

Embodiments of the disclosure include methods and compositions for the treatment of any medical condition related to the mammalian heart. In specific embodiments, the disclosure concerns treatment of one or more cardiac medical conditions with therapeutic compositions that affect endogenous cells or tissue in the heart. In particular embodiments, therapy is provided to an individual in need thereof, such as when the individual has a need for in situ or in vivo therapy of endogenous cardiac tissue because of a cardiac medical condition or risk thereof. In specific embodiments, the individual has cardiac cellular or cardiac tissue damage from a cardiac medical condition.

In certain embodiments, the disclosure improves upon existing methods and compositions for cardiac medicine by improving the efficiency of cardiomyocyte production over methods compared to the absence of the methods and compositions of the disclosure. In specific cases, the disclosure concerns enhancement of a pre-cardiomyocyte transdifferentiation step by improving upon the type of cell upon which the transdifferentiation to the cardiomyocyte occurs. In specific cases, the cells that are subject to transdifferentiation to cardiomyocytes are not the same cells in existing methods of transdifferentiation to cardiomyocytes. In particular cases, the cells that are subject to transdifferentiation to cardiomyocytes are not fibroblasts, as in existing methods.

In particular embodiments, methods and compositions of the disclosure utilize fibroblasts, including cardiac fibroblasts, as an initial source of cells but instead of subjecting the fibroblasts to transdifferentiation to cardiomyocytes the fibroblasts are first converted to endothelial cells or endothelial-like cells (for example, endothelial-like cells, having some but not necessarily all endothelial cell features (e.g., expressing markers like Factor VIII or PECAM-1, FLI1, ERG, VE-Cadherin, ESM1, KDR, or CXCL12), and this occurs as an intended, active step of the method. In certain embodiments, fibroblasts are modified by being exposed to one or more compositions, and this modification converts the fibroblasts to endothelial cells or endothelial-like cells, upon which transdifferentiation to cardiomyocytes occurs.

Particular embodiments of the disclosure encompass methods whereby early administration with one or more compositions improves the efficiency of direct reprogramming of cardiac fibroblasts into cardiomyocytes through an intermediate, other type of cell. In certain cases, the methods encompass exposing fibroblasts to a differentiating factor to improve the efficiency of direct reprogramming of cardiac fibroblasts into cardiomyocytes through an intermediate, other type of cell. In specific embodiments, the differentiating factor is Ets variant 2 (ETV2) and/or VEGF that improves the efficiency of direct reprogramming of cardiac fibroblasts into cardiomyocytes by producing an intermediate type of cell first. In specific embodiments, endothelial cells or endothelial-like cells are produced upon exposure of ETV2 and/or VEGF to fibroblasts, and the endothelial cells or endothelial-like cells are the subject of reprogramming to cardiomyocytes.

The disclosed methods improve upon earlier cardiac reprogramming studies that demonstrated that administration of three transcription factors (Gata4, Mef 2c and Tbx5, collectively referred to as GMT) could directly transform cardiac fibroblasts into cardiomyocyte-like cells (iCMs). However, the reprogramming efficiency of the GMT cocktail method remains low. In the disclosed methods embodied herein, prior infection of cardiac fibroblasts with inducible ETV2 and/or VEGF lentivirus (or otherwise exposure to) before GMT administration to the fibroblasts facilitated transdifferentiation of cardiac fibroblasts into endothelial progenitors and significantly enhanced the differentiation efficiency of these cells into cardiomyocytes by GMT in vitro.

Thus, embodiments of the disclosure encompass the targeting of endothelial cells or endothelial-like cells as a cardiomyocyte source. The disclosure includes methods in which endothelial cells or endothelial-like cells (generated from fibroblasts transfected with or otherwise exposed to ETV2 and/or VEGF) are reprogrammed into cardiomyocytes with one or more transdifferentiation factors that may or may not include part or all of GMT.

Embodiments of the disclosure include direct reprogramming of cardiac fibroblasts into cardiomyocytes using an endothelial cell transdifferentiation strategy. Embodiments of the disclosure include methods of producing cardiomyocytes, comprising the step of exposing ETV2- and/or VEGF-transfected fibroblasts, ETV2- and/or VEGF-transfected endothelial cells or endothelial-like cells, or two or more of these, to one or more cardiomyocyte transdifferentiation factors, thereby producing the cardiomyocytes. Embodiments of the disclosure include methods of producing cardiomyocytes, comprising the step of exposing ETV2- and/or VEGF-expressing fibroblasts, ETV2- and/or VEGF-expressing endothelial cells or endothelial-like cells, or both, to one or more cardiomyocyte transdifferentiation factors, thereby producing the cardiomyocytes. Embodiments of the disclosure include methods of producing cardiomyocytes, comprising the step of exposing ETV2- and/or VEGF-expressing endothelial cells or endothelial-like cells, and optionally ETV2- and/or VEGF-expressing fibroblasts, to one or more cardiomyocyte transdifferentiation factors, thereby producing the cardiomyocytes. The method may occur in vivo or ex vivo.

Specific embodiments provide for converting fibroblasts into endothelial cells or endothelial-like cells to enhance their susceptibility to reprogramming into cardiomyocytes as a cardiac regeneration strategy. The endothelial cells or endothelial-like cells are a cardiomyocyte reprogramming target, in specific aspects of the disclosure. Fibroblast reprogramming into endothelial cells or endothelial-like cells may be used to increase the “supply” of endothelial cells or endothelial-like cells as a transition state for fibroblast to cardiomyocyte reprogramming.

As shown herein, and in specific cases, infection of cardiac fibroblasts with inducible ETV2- and/or VEGF-lentivirus prior to GMT administration facilitated transdifferentiation of cardiac fibroblasts into endothelial progenitors and significantly enhanced the differentiation efficiency of these cells into cardiomyocytes by GMT in vitro, as evidenced by one example of a lineage marker expression profile.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 shows an illustration of one embodiment of enhanced reprogramming via endothelial cell transition. It illustrates the rationale for endothelial cell transition targeting as a cardio-differentiation strategy.

FIG. 2 shows that endothelial cells can be transdifferentiated into cardiomyocytes at a higher rate fibroblasts. GMT-treated cardiac fibroblasts demonstrated less cardiac troponin expression versus GMT-treated endothelial cells, in which the green bars (left in each pair) did not receive GMT and the blue bars (right in each pair) received GMT. *: p<0.05; **: p<0.01.

FIG. 3 shows that ETV2 can transdifferentiate fibroblasts into endothelial cells. Expression of endothelial lineage markers, KDR, ERG, and FLI1 in ETV-infected cells is shown. Data is shown as relative fold to no ETV2 group.

FIG. 4 demonstrates cardio-differentiation of transdifferentiated endothelial cells versus fibroblasts. In vitro cardiomyocyte marker expression (cTnT) after initial treatment of cardiac fibroblasts with ETV2, followed by exposure to the GMT cardio-differentiating factors is shown.

FIG. 5 provides one example timeline for of an in vivo Experimental Design for a rat cornonary ligation model in which rats exposed to ETV2 prior to GMT treatment are compared to control rats not exposed to ETV2 prior to GMT treatment.

FIG. 6 demonstrates echocardiographic analysis of ejection fraction following ETV2 versus ETV2/GMT therapy in a rat coronary ligation model. The change of the cardiac function marker, ejection fraction (EF), between ETV2 and no ETV2 at the time of GMT injection (left graph) and at the time of euthanasia is shown (right graph). The left ventricular (LV) end-systolic and end-diastolic diameters and anterior and posterior wall thickness were measured from M-mode tracings acquired at the level of the papillary muscle. Each animal received echocardiographyic assessments 4 times, pre-first surgery, day 3 after the first surgery, pre-second surgery, and day 28 after the second surgery (see FIG. 5).

FIG. 7 shows (7A) a schematic of in vitro testing protocol for simultaneous treatment of cardiac fibroblasts with VEGF or ETV2 and Gata4, Mef2c and Tbx % (GMT). “Dox” indicates doxycycline-mediated activation of ETV2. (7B) Results for treatments depicted in (7A), using qPCR analysis for the cardiomyocyte marker cTnT, demonstrating that simultaneous VEGF+GMT treatment of cells is superior to simultaneous ETV2+GMT treatment, and that pre-treatment of cells with VEGF yielded similar subsequent cardio-differentiation efficiency as induced by ETV2 pre-treatment.

FIG. 8 shows (8A) a schematic of in vitro testing protocol for sequential treatment of cardiac fibroblasts with VEGF or ETV2 and Gata4, Mef2c and Tbx % (GMT). “Dox” indicates doxycycline-mediated activation of ETV2. (8B) Results for treatments depicted in (8A), using qPCR analysis for the cardiomyocyte marker cTnT, demonstrating that sequential VEGF+GMT treatment of cells is superior to GMT treatment alone, and that pre-treatment of cells with VEGF yielded similar subsequent cardio-differentiation efficiency as induced by ETV2 pre-treatment.

### DETAILED DESCRIPTION

This application incorporates by reference herein in its entirety U.S. Provisional Patent Application Ser. No. 62/819,636, filed Mar. 17, 2019, and U.S. Provisional Patent Application Ser. No. 62/830,543, filed Apr. 7, 2019.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, “differentiation” (e.g., cell differentiation) describes a process by which an unspecialized (or “uncommitted”) or less specialized cell acquires the features (e.g., gene expression, cell morphology, etc.) of a specialized cell, such as a nerve cell or a muscle cell for example. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, “transdifferentiation” describes a process by which one cell type differentiates into a different cell type or reverts to a less differentiated cell type. In some embodiments of the disclosure, “transdifferentiation” of fibroblasts to cardiomyoctes is described.

As used herein, the term “therapeutically effective amount” is synonymous with “effective amount”, “therapeutically effective dose”, and/or “effective dose” refers to an amount of an agent sufficient to ameliorate at least one symptom, behavior or event, associated with a pathological, abnormal or otherwise undesirable condition, or an amount sufficient to prevent or lessen the probability that such a condition will occur or re-occur, or an amount sufficient to delay worsening of such a condition. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

### I. General Embodiments

In development, endothelial cells, vascular smooth muscle cells, and cardiomyocytes are all differentiated from a common progenitor in the mesoderm. Furthermore, endothelial cells are well known to have the ability to enter a process called Endothelial Mesenchymal Transition (EndMT), during which endothelial cells exhibit remarkable phenotypic plasticity. In contrast to nearly all previous strategies that have remained focused on the fibroblast as the target cell for generating induced cardiomyocytes (iCM), it was considered and is encompassed herein that reprogramming fibroblasts towards endothelial cells will yield high plasticity and a pathway to efficient cardiomyogenic transdifferentiation.

An in vivo application of the strategy that endothelial cell reprogramming into iCM is potentially limited by the critical role of endothelial cells as vascular constituents and the relative scarcity of these as target cells, as compared to the preferred fibroblast cell target. Encompassed in this disclosure is the contemplation that reprogramming of fibroblasts into endothelial cells or endothelial-like cells as the primary target of this transdifferention strategy would generate an endothelial “meso” stage in a novel fibroblast-to-endothelial cell-to-iCM pathway. This “two hit” approach would provide the added advantage of preventing uncontrolled endothelial cell proliferation and potential hemangioma formation. Therefore, embodiments of the disclosure encompass endothelial cell “meso” staging to enhance iCM generation.

As shown herein, the inventors leverage evidence that the reprogramming of fibroblasts into endothelial cells or endothelial-like cells could be accomplished via the vascular endothelial cell master regulator ETV2 and/or VEGF as a means to demonstrate this EC meso reprogramming strategy. The inventors first demonstrated that ETV2 and/or VEGF induced transdifferentiation of endothelial-like cells and EndMT in cardiac fibroblasts (Fibroblast-Endothelial-Mesenchymal cell Transition). Next, the inventors performed cardiac fibroblasts reprogramming into cardiomyocytes by inducing ETV2 and/or VEGF factor prior to GMT introduction that resulted in higher efficiency of iCM cell production in vitro compared with GMT alone.

As encompassed herein, cardiac microvascular endothelial cells were transdifferentiated into cardiomyocyte-like cells (iCMs) by GMT with much higher efficiency than were cardiac fibroblasts. The disclosure encompasses the novel strategy of differentiating cardiac fibroblasts into endothelial-like cells as an enhanced precursor to iCM generation. This strategy can be applied as an in situ strategy of myocardial regeneration using direct delivery of genetic factors into ischemic/infarcted myocardium as a mean of relieving heart failure without the need to inject exogenous (stem) cells, which is being identified as an ineffective regeneration strategy.

Embodiments of the disclosure encompass methods having at least two steps: generation of endothelial cells or endothelial-like cells from fibroblasts upon exposure of fibroblasts to one or more particular differentiating factors followed by generation of cardiomyocytes from the endothelial cells or endothelial-like cells upon exposure of the endothelial cells to one or more particular transdifferentiation factors. Thus, in specific embodiments, there are methods that require generation of endothelial cells or endothelial-like cells prior to generation of cardiomyocytes.

In particular embodiments, delivery of certain composition(s) to cells in situ or in vivo in the individual allows regeneration of cardiac tissue by allowing reprogramming of endogenous non-cardiomyocyte cells, such as fibroblasts, to become cardiomyocytes. Upon delivery of a therapeutically effective amount of one or more composition(s) to the individual, the composition(s) provide improvement of the condition at least in part, such as by allowing regeneration of cardiac tissue or cells therein. In specific embodiments, the composition(s) comprise ETV2 and/or VEGF and one or more transdifferentiation factors. In specific cases, ETV2 and/or VEGF and the one or more transdifferentiation factors are provided to the individual at the same time, whereas in other cases ETV2 and/or VEGF and the one or more transdifferentiation factors are provided to the individual sequentially, with ETV2 and/or VEGF provided to the individual prior to the one or more transdifferentiation factors.

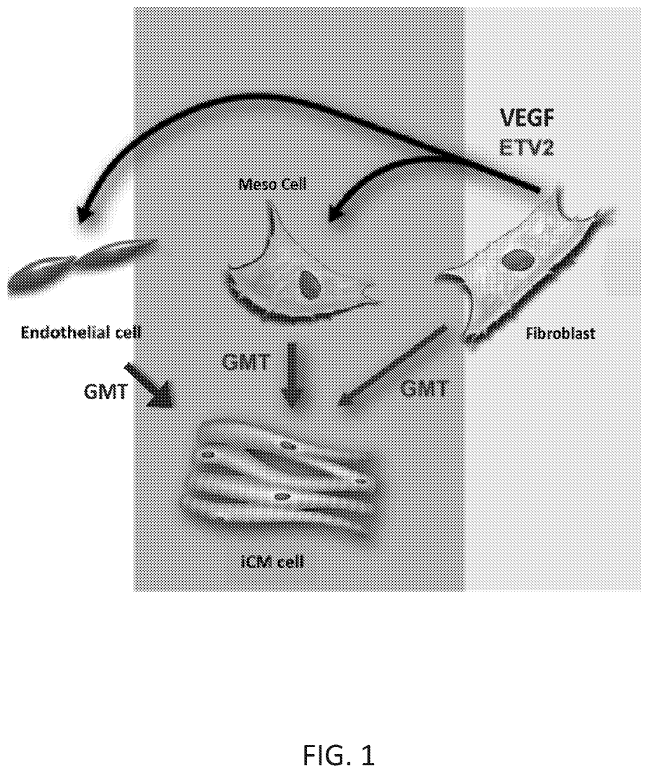

As illustrated in FIG. 1, endothelial cell “Meso” staging enhances iCM generation. FIG. 1 illustrates one embodiment for cell phenotypic changes with methods of the disclosure. ETV2 and/or VEGF induces Fibroblast-Endothelial Transition, and those endothelial-like cells have higher plasticity and generate more iCM cells with GMT (or other differentiated cells with their respective differentiation factor(s)).

Embodiments of the disclosure encompass methods of producing differentiated cells from fibroblasts for an individual, comprising the steps of (a) subjecting fibroblasts to an effective amount of ETV2 and/or VEGF to produce endothelial cells or endothelial-like cells; and (b) subjecting the endothelial cells or endothelial-like cells to an effective amount of one or more transdifferentiation factors to produce the differentiated cells. Steps (a) and (b) occur in vivo or in vitro. When the method occurs in vivo, the ETV2 and/or VEGF and the one or more transdifferentiation factors may be provided to the individual at substantially the same time. In other cases, the ETV2 and/or VEGF may be provided to the individual prior to providing the one or more transdifferentiation factors to the individual. In some cases, the method occurs in vitro, the ETV2 and/or VEGF and the one or more transdifferentiation factors are provided to a culture comprising fibroblasts at substantially the same time. In other cases, when the method occurs in vitro, the ETV2 and/or VEGF is provided to a culture comprising fibroblasts prior to providing the one or more transdifferentiation factors to the culture.

In particular embodiments, an in vivo method is utilized to produce cardiomyocytes in an individual. In such cases, the ETV2 and/or VEGF and the one or more transdifferentiation factors are provided to the individual, and the production of endothelial cells or endothelial-like cells and the subsequent production of cardiomyocytes occurs in vivo. In specific embodiments, the ETV2 and/or VEGF and the one or more transdifferentiation factors are provided to the individual in either polynucleotide or polypeptide form, and the delivery may be systemic or local. In local delivery, the ETV2 and/or VEGF and the one or more transdifferentiation factors may be provided directly to the site of infarction (and the site may include or be a scar). In cases wherein the ETV2 and/or VEGF and the one or more transdifferentiation factors are provided systemically to the individual, the ETV2 and/or VEGF and the one or more transdifferentiation factors may include targeting agents. Examples of targeting agents include AAV vectors, for example an AAV vector serotype 9 that has predilection for cardiac cells. The vector may also comprise a regulatable promoter that only allows expression in appropriate cells (e.g., fibroblast-specific promoters that target fibroblasts).

Particular embodiments of the disclosure encompass methods of in vivo reprogramming of cardiac cells in an individual, comprising the step of providing locally to the heart of the individual a therapeutically effective amount of (a) ETV2 and/or VEGF; and (b) one or more transdifferentiation factors, wherein the one or more transdifferentiation factors are provided to the individual at the same time or after providing the ETV2 and/or VEGF to the individual. In specific embodiments, the individual has had a myocardial infarction and the ETV2 and/or VEGF and one or more transdifferentiation factors are provided at a location in the heart that was damaged by the myocardial infarction, for example a location in the heart that has scar tissue.

### II. Generation of Endothelial Cells or Endothelial-Like Cells from Fibroblasts

Embodiments of the disclosure encompass methods in which fibroblasts are utilized as a de novo source of endothelial cells. In specific embodiments, fibroblasts are differentiated into endothelial cells or endothelial-like cells by one or more differentiating factors, such as ETV2 and/or VEGF. In particular embodiments, the fibroblasts are exposed to an effective amount of ETV2 and/or VEGF upon transfection of the fibroblasts with a vector that encodes ETV2 and/or VEGF, although in alternative embodiments the fibroblasts are exposed to a sufficient amount of externally provided ETV2 and/or VEGF gene product.

The generation of endothelial cells or endothelial-like cells from fibroblasts may occur in vivo or ex vivo. In cases wherein fibroblasts are differentiated to endothelial cells or endothelial-like cells by ETV2 and/or VEGF in an in vivo setting, an effective amount of ETV2 and/or VEGF may be delivered in the form of a polynucleotide and/or polypeptide to endogenous fibroblasts located in vivo, such as cardiac fibroblasts present in the heart of an individual. In such cases, the ETV2 and/or VEGF may be delivered in a suitable carrier, such as liposomes, nanoparticles, by direct injection (including into the myocardium), for example via a needle, into endocardium via catheter, into epicardium via trans-thoracic procedure, intravascularly with targetable agent, etc. In cases wherein fibroblasts are differentiated to endothelial cells or endothelial-like cells by ETV2 and/or VEGF in an ex vivo setting, the fibroblasts may be exposed to an effective amount of ETV2 and/or VEGF polynucleotide and/or polypeptide, such as in culture. Following exposure to ETV2 and/or VEGF, the fibroblasts may then be delivered to the heart of the individual. In addition, or alternatively, in an ex vivo setting the fibroblasts may be transfected with ETV2 and/or VEGF on a vector and the fibroblasts express ETV2 and/or VEGF; following transfection the fibroblasts may then be delivered to the heart of the individual.

In cases wherein ETV2 and/or VEGF is present on a vector, the vector may be viral or non-viral. Examples of non-viral vectors include plasmids, transposons, and the like. Examples of viral vectors include lentiviral, adenoviral, adeno-associated, or retroviral vectors. The expression of the ETV2 and/or VEGF may be controlled by one or more regulatory elements, including promoters and/or enhancers. One or more regulatory elements may be tissue-specific, inducible, constitutive, and so forth. Examples of fibroblast-specific promoters include, for example, periostin and FSP1.

The ETV2 and/or VEGF gene and gene product is utilized in methods of the disclosure. Other names for ETV2 include ETS Variant 2, ER71, and ETSRP71. Other names for VEGF include vascular permeability factor (VPF). In some examples, an ETV2 and/or VEGF polynucleotide is delivered to an individual in need thereof, whether it be in the form of being on a vector, associated with a carrier, within a cell (including in a cell on a vector), and so forth. In specific embodiments, the ETV2 and/or VEGF polynucleotide is a mammalian ETV2 and/or VEGF polynucleotide, including human, mouse, rat, and so forth.

One example of an ETV2 polynucleotide sequence is in the GenBank® Accession No. NM_001300974 (SEQ ID NO:1):

| | |
|---|---|
| 1 | ttcctgttgc agataagccc agcttagccc agctgacccc |
| | agaccctctc ccctcactcc |
| 61 | ccccatgtcg caggatcgag accctgaggc agacagcccg |
| | ttcaccaagc cccccgcccc |
| 121 | gcccccatca ccccgtaaac ttctcccagc ctccgccctg |
| | ccctcaccca gcccgctgtt |
| 181 | ccccaagcct cgctccaagc ccacgccacc cctgcagcag |
| | ggcagcccca gaggccagca |
| 241 | cctatccccg aggctggggt cgaggctcgg ccccgcccct |
| | gcctctgcaa cttgagcctg |
| 301 | gctgcgaccc ctgctctgac gtctcggaaa attccccctt |
| | gcccaggccc ttgggggagg |
| 361 | gggtgcatgg tatgaaatgg ggctgagacc cccggctggg |
| | ggcagaggaa cccgccagag |
| 421 | aaggagccaa attaggcttc tgtttccctg atctggcact |
| | ccaaggggac acgccgacag |
| 481 | cgacagcaga gacatgctgg aaaggtacaa gctcatccct |
| | ggcaagcttc ccacagctgg |
| 541 | actggggctc cgcgttactg cacccagaag ttccatgggg |
| | ggcggagccc gactctcagg |
| 601 | ctcttccgtg gtccggggac tggacagaca tggcgtgcac |
| | agcctgggac tcttggagcg |
| 661 | gcgcctcgca gaccctgggc cccgcccctc tcggcccggg |
| | ccccatcccc gccgccggct |
| 721 | ccgaaggcgc cgcgggccag aactgcgtcc ccgtggcggg |
| | agaggccacc tcgtggtcgc |
| 781 | gcgcccaggc cgccgggagc aacaccagct gggactgttc |
| | tgtggggccc gacggcgata |
| 841 | cctactgggg cagtggcctg ggcggggagc cgcgcacgga |
| | ctgtaccatt tcgtggggcg |
| 901 | ggcccgcggg cccggactgt accacctcct ggaacccggg |
| | gctgcatgcg ggtggcacca |
| 961 | cctctttgaa gcggtaccag agctcagctc tcaccgtttg |
| | ctccgaaccg agcccgcagt |
| 1021 | cggaccgtgc cagtttggct cgatgcccca aaactaacca |
| | ccgaggtccc attcagctgt |
| 1081 | ggcagttcct cctggagctg ctccacgacg gggcgcgtag |
| | cagctgcatc cgttggactg |
| 1141 | gcaacagccg cgagttccag ctgtgcgacc ccaaagaggt |
| | ggctcggctg tggggcgagc |
| 1201 | gcaagagaaa gccgggcatg aattacgaga agctgagccg |
| | gggccttcgc tactactatc |
| 1261 | gccgcgacat cgtgcgcaag agcggggggc gaaagtacac |
| | gtaccgcttc gggggccgcg |
| 1321 | tgcccagcct agcctatccg gactgtgcgg gaggcggacg |
| | gggagcagag acacaataaa |
| 1381 | aattcccggt caaacctcaa aaaaaaaaaa aaa |

One example of a VEGF polynucleotide sequence is in the GenBank® Accession No. AY047581 (SEQ ID NO:2)

| | |
|---|---|
| 1 | tcgggcctcc gaaaccatga actttctgct gtcttgggtg cattggagcc ttgccttgct |
| 61 | gctctacctc caccatgcca agtggtccca ggctgcaccc atggcagaag gaggggggca |
| 121 | gaatcatcac gaagtggtga agttcatgga tgtctatcag cgcagctact gccatccaat |
| 181 | cgagaccctg gtggacatct tccaggagta ccctgatgag atcgagtaca tcttcaagcc |
| 241 | atcctgtgtg cccctgatgc gatgcggggg ctgctgcaat gacgagggcc tggagtgtgt |
| 301 | gcccactgag gagtccaaca tcaccatgca gattatgcgg atcaaacctc accaaggcca |
| 361 | gcacatagga gagatgagct tcctacagca caacaaatgt gaatgcagac caaagaaaga |
| 421 | tagagcaaga caagaaaatc cctgtgggcc ttgctcagag cggagaaagc atttgtttgt |
| 481 | acaagatccg cagacgtgta aatgttcctg caaaaacaca gactcgcgtt gcaaggcgag |
| 541 | gcagcttgag ttaaacgaac gtacttgcag atgtgacaag ccgaggcggt gagccgggca |
| 601 | ggaggaagga gcctccctca gggtttcggg aaccagatct |

In particular embodiments, part or all of SEQ ID NO:1 and/or SEQ ID NO:2 is utilized in methods of the disclosure. In specific embodiments, a polynucleotide having a specific sequence identity with respect to SEQ ID NO:1 and/or SEQ ID NO:2 is utilized in methods of the disclosure. In specific cases, a functional fragment of SEQ ID NO:1 and/or SEQ ID NO:2 is employed, and the term “functional fragment” as used herein refers to a polynucleotide that encodes a polypeptide having the activity of being able to convert fibroblasts to endothelial cells or endothelial-like cells. In specific cases, the fragment has a length of at least about or no more than about 1375, 1350, 1325, 1300, 1275, 1250, 1225, 1200, 1175, 1150, 1125, 1100, 1075, 1050, 1025, 1000, 975, 950, 925, 900, 875, 850, 825, 800, 775, 750, 725, 700, 675, 650, 625, 600, 575, 550, 525, 500, 475, 450, 425, 400, 375, 350, 325, 300, 275, 250, 225, 200, 175, 150, 125, or 100 contiguous nucleotides of SEQ ID NO:1 and/or SEQ ID NO:2. In addition, the fragment may have sequence identity with the corresponding region in SEQ ID NO:1 and/or SEQ ID NO:2 of 100, 99, 98, 97, 96, 95, 94, 93, 92, 91, 90, 89, 88, 87, 86, 85, 80, 75, or 70% identity. A polynucleotide having certain sequence identity to SEQ ID NO:1 and/or SEQ ID NO:2 may be used, including 99, 98, 97, 96, 95, 94, 93, 92, 91, 90, 89, 88, 87, 86, 85, 80, 75, or 70% identity to SEQ ID NO:1 and/or SEQ ID NO:2.

In some examples, an ETV2 and/or VEGF polypeptide is delivered to an individual in need thereof, whether it be in the form of being on a vector, associated with a carrier, within a cell (including in a cell on a vector), and so forth. In specific embodiments, the ETV2 and/or VEGF polypeptide is a mammalian ETV2 and/or VEGF polypeptide, including human, mouse, rat, and so forth. In particular embodiments, one example of an ETV2 polypeptide sequence is in the GenBank® Accession No. NP_001287903 (SEQ ID NO:3):

| | |
|---|---|
| 1 | mactawdsws gasqtlgpap lgpgpipaag segaagqncv |
| | pvageatsws raqaagsnts |
| 61 | wdcsvgpdgd tywgsglgge prtdctiswg gpagpdctts |
| | wnpglhaggt tslkryqssa |
| 121 | ltvcsepspq sdraslarcp ktnhrgpiql wqfllellhd |
| | garsscirwt gnsrefqlcd |
| 181 | pkevarlwge rkrkpgmnye klsrglryyy rrdivrksgg |
| | rkytyrfggr vpslaypdca |
| 241 | gggrgaetq |

In particular embodiments, one example of a VEGF polypeptide sequence is in the GenBank® Accession No. AAK95847 (SEQ ID NO:4):

| | |
|---|---|
| 1 | mnfllswvhw slalllylhh akwsqaapma egggqnhhev |
| | vkfmdvyqrs ychpietivd |
| 61 | ifqeypdeie yifkpscvpl mrcggccnde glecvptees |
| | nitmqimrik phqgqhigem |
| 121 | sflqhnkcec rpkkdrarqe npcgpcserr khlfvqdpqt |
| | ckcsckntds rckarqleln |
| 181 | ertcrcdkpr r |

In particular embodiments, part or all of SEQ ID NO:3 and/or SEQ ID NO:4 is utilized in methods of the disclosure. In specific embodiments, a polypeptide having a specific sequence identity with respect to SEQ ID NO:3 and/or SEQ ID NO:4 is utilized in methods of the disclosure. In specific cases, a functional fragment of SEQ ID NO:3 and/or SEQ ID NO:4 is employed, and the term “functional fragment” as used herein refers to a polypeptide having the activity of being able to convert fibroblasts to endothelial cells or endothelial-like cells. In specific cases, the fragment has a length of at least about or no more than about 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25, or 20 contiguous amino acids of SEQ ID NO:3 and/or SEQ ID NO:4.

Embodiments of the disclosure include generating an endothelial cell “meso” stage in an “induced cardiomyocytes” (iCM) pathway in which case iCMs are produced from the endothelial cells or endothelial-like cells.

In cases wherein ETV2 and/or VEGF is delivered to endogenous fibroblasts in the heart of an individual in need thereof, the delivery method may be local and may be delivered by any suitable method directly to the heart. The local delivery may be by injection, by stent delivery, a balloon-based delivery, echo-guided injection from inside the cardiac cavity, or placement of patch or gel comprising ETV2 and/or VEGF on the scar, for example. The local delivery may or may not occur in the heart at a location of cardiac tissue in need, including diseased and/or damaged cardiac tissue. In specific embodiments, the damaged cardiac tissue is damaged from an infarct. The local delivery may be a single delivery, or there may be multiple deliveries over time, such as over the course of 1-7 days, 1-4 weeks, 1-12 months or one or more years.

In cases wherein ETV2 and/or VEGF is delivered to fibroblasts ex vivo, the fibroblasts may be autologous, allogeneic, or xenogeneic with respect to the recipient individual. Although in particular embodiments the fibroblasts are cardiac fibroblasts, in other embodiments the fibroblasts are derived from a source of tissue selected from the group consisting of: a) adipose; b) dermal; c) placental; d) hair follicle; e) keloid; f) bone marrow; g) peripheral blood; h) umbilical cord; i) foreskin; j) omentum; and k) a combination thereof. The fibroblasts may be transfected with ETV2 and/or VEGF on a vector and may be delivered to the individual in any suitable manner, including locally, such as by injection and/or within a stent and/or balloon. In some cases, the fibroblasts are stored prior to delivery to an individual.

Although ex vivo the fibroblasts may be transfected with ETV2 and/or VEGF, in other embodiments the fibroblasts are exposed to ETV2 and/or VEGF that is exogenously provided, such as exposed to upon culture of the fibroblasts with a sufficient amount of ETV2 and/or VEGF in the media of the culture. The culture of fibroblasts with ETV2 and/or VEGF may occur over a sufficient period of time, including over the course of one or more passages of the culture. The media may be changed to provide fresh amounts of ETV2 and/or VEGF or change the concentration of the ETV2 and/or VEGF. The exposure of the fibroblasts to ETV2 and/or VEGF may be monitored, for example an aliquot of the culture may be obtained and tested whether the cells therein have one or more expression markers associated with endothelial cells.

The ETV2- and/or VEGF-transfected fibroblasts and/or ETV2- and/or VEGF-exposed fibroblasts may be sold commercially. The ETV2- and/or VEGF-transfected fibroblasts and/or ETV2- and/or VEGF-exposed fibroblasts may be stored and/or sold in a delivery device, such as a syringe, stent, or balloon, as examples only.

In certain embodiments, following delivery of an effective amount of ETV2 and/or VEGF to the heart of an individual (whether or not delivered in fibroblasts or without fibroblasts), there may or may not be assessment whether endothelial cells or endothelial-like cells are produced or monitoring of the production of the endothelial cells or endothelial-like cells. Cardiac tissue from the individual may be assayed for one or more particular markers of endothelial cells or endothelial-like cells. In some cases, the individual may be monitored by standard means to identify if there is improvement of cardiac tissue following delivery of the ETV2 and/or VEGF (and subsequent to delivery of one or more transdifferentiation factors to cardiomyocytes).

Following delivery of an effective amount of ETV2 and/or VEGF to an individual, and/or ETV2- and/or VEGF-transfected fibroblasts and/or ETV2- and/or VEGF-exposed fibroblasts, endothelial cells or endothelial-like cells are produced and the individual is provided an effective amount of one or more transdifferentiation factors for production of cardiomyocytes.

### III. Generation of Differentiated Cells from Endothelial Cells

Following production of endothelial cells or endothelial-like cells upon exposure of fibroblasts to ETV2 and/or VEGF, the produced endothelial cells or endothelial-like cells are utilized as a substrate for producing or regenerating differentiated cells of a desired cell type. The differentiated cells of a desired cell type may be of any kind, and the one or more transdifferentiation factors may be selected based upon the desired cell type. In specific cases, the differentiated cells are cardiomyocytes, hepatocytes, adipocytes, neural cells (including neurons), pancreatic cells (including pancreatic beta cells), skeletal myocytes, chondrocytes, or osteoblasts, for example. In specific embodiments, the endothelial cells or endothelial-like cells are utilized as a substrate for producing or regenerating differentiated cells rather than producing the differentiated cells directly from fibroblasts that have been exposed to ETV2 and/or VEGF (including upon transfection within the fibroblasts or upon exposure to exogenously provided ETV2 and/or VEGF).

In particular embodiments, the endothelial cells or endothelial-like cells are differentiated into cardiomyocytes upon exposure of the endothelial cells or endothelial-like cells to one or more transdifferentiation factors. The transdifferentiation factor(s) may be of any suitable kind that allows differentiation of the endothelial cells or endothelial-like cells to cardiomyocytes, but in specific embodiments, the one or more transdifferentiation factors for differentiation into any type of cell are transcription factors. The transcription factors may regulate expression of one or more genes that directly or indirectly initiate or are otherwise involved in differentiation to the desired cell. In the example case of cardiomyocytes, the transcription factor may directly or indirectly regulate expression of one or more specific markers associated with cardiomyocytes (for example, cardiac troponin C, Alpha actinin (Actc1), cardiac myocin heavy chain (MYH7), and so forth). In any event, the one or more transcription factors may be selected for being active during the development of the desired differentiated cell type or for directing the differentiation of fibroblasts, endothelial cells, and/or endothelial-like cells into a specific differentiated cell type.

The transdifferentiation factor(s) may be subjected to the endothelial cells in any suitable manner. In specific embodiments, transdifferentiation occurs for the endothelial cells (including endothelial cells produced following exposure of fibroblasts to ETV2 and/or VEGF) upon subjecting the endothelial cells to the following: (1) exposure of the endothelial cells to vector(s) encoding the one or more transdifferentiation factors; (2) introducing exogenous transgenes into the endothelial cells that encode the one or more transdifferentiation factors (3) genetically engineering endogenous genes in the endothelial cells (for example, silencing one or more genes), such as by CRISPR/Cas9; (4) exposing the endothelial cells to one or more pharmacological agents; or (5) a combination thereof.

In specific embodiments related to the production of cardiomyocytes, the one or more transdifferentiation factors utilized in methods of the disclosure are selected from the group consisting of Gata4 (also known as: ASD2, TACHD, TOF, VSD1), Mef2c, Tbx5, ETV2, VEGF, myocardin, Hand2, myocardin, miRNA-590, p63shRNA, Mesoderm posterior protein 1 (Mesp1), miR-133, miR-1, Oct4, Klf4, c-myc, Sox2, Brachyury, Nkx2.5, ETS2, ESRRG, Mrtf-A, MyoD, ZFPM2, 5-azacytidine, Zebularine, miRNA-1, miRNA-133, miRNA-208, miRNA-499, or a combination thereof. In specific embodiments, the one or more transdifferentiation factors utilized for production of cardiomyocytes in the methods are Gata4, Mef2c, and Tbx5, although in alternative embodiments one or more of Gata4, Mef2c, Tbx5 are not utilized. In particular embodiments, one or more of Gata4, Mef2c, Tbx5, ETV2, VEGF, Hand2 and myocardin are utilized.

In specific embodiments related to the production of neurons, the one or more transdifferentiation factors utilized in methods of the disclosure are selected from the group consisting of Brn2, Mty1l, miRNA-124, Ascl1, Brn2, Myt1l, Ngn2, Ascl1, Brn2, Dimethylsulphoxide, butylated hydroxy-anisole, KCl, valproic acid, forskolin, hydrocortisone, insulin, and a combination thereof.

In specific embodiments related to the production of hepatocytes, the one or more transdifferentiation factors utilized in methods of the disclosure are selected from the group consisting of Foxa2, Hnf4α, C/EBPβ, c-Myc, Hnf1α, Hnf4α, Foxa3, Dexamethasone, oncostatin M, and a combination thereof.

In specific embodiments related to the production of skeletal myocytes, the one or more transdifferentiation factors utilized in methods of the disclosure are selected from the group consisting of 5-azacytidine, Myod1, SB431542, Chir99021, EGF, IGF1, and a combination thereof.

In specific embodiments related to the production of chondrocytes, the one or more transdifferentiation factors utilized in methods of the disclosure are selected from the group consisting of Cartilage-derived morphogenetic protein 1, c-Myc, KLF4, Sox9, and a combination thereof.

In specific embodiments related to the production of pancreatic beta cells, the one or more transdifferentiation factors utilized in methods of the disclosure are selected from the group consisting of Pdx1, Ngn3, Mafa, MAPK, STATS, and a combination thereof.

In specific embodiments related to the production of adipocytes, the one or more transdifferentiation factors utilized in methods of the disclosure are selected from the group consisting of Myod1, Dexamethasone, 1-methyl-3-isobutylxanthine, PPARγ agonists, and a combination thereof.

In specific embodiments related to the production of osteoblasts, the one or more transdifferentiation factors utilized in methods of the disclosure are selected from the group consisting of Calcitriol, dexamethasone, ascorbic acid, and beta-glycerophosphate, Runx2, MKP-1, and a combination thereof.

In specific embodiments, when more than one transdifferentiation factor is utilized, they may be provided to the individual at the same time or at different times. They may be provided to the individual in the same composition or in different compositions.

In some examples, transdifferentiation factor(s) is delivered to an individual in need thereof in the form of a polynucleotide or a polypeptide. The factor may be delivered on a vector, associated with a carrier, within a cell (including in a cell on a vector), and so forth. In specific embodiments, the transdifferentiation factor(s) is a mammalian transdifferentiation factor(s), including human, mouse, rat, and so forth.

In some embodiments, transdifferentiation factor nucleic acids are comprised on separate vectors or on the same vector. In certain cases, the vector is a viral vector or a non-viral vector, such as a nanoparticle, plasmid, liposome, or a combination thereof. In a specific embodiment, the viral vector is an adenoviral, lentiviral, retroviral, adeno-associated viral vector, or episomal (non-integrating) vectors. In specific embodiments, any of the compositions herein may be delivered encapsulated in liposomes, by iontophoresis, or by incorporation into other vehicles such as hydrogels, cyclodextrins, biodegradable nanocapsules, and bioadhesive microspheres. The transdifferentiation factor nucleic acids may be provided to the recipient cells through non-integrating, non-viral methods such as transient transfection and/or electroporation.

The transdifferentiation factor-encoding (and/or ETV2- and/or VEGF-encoding) nucleic acids of the present disclosure can be formulated in pharmaceutical compositions, which are prepared according to conventional pharmaceutical compounding techniques. See, e.g., Remington's Pharmaceutical Sciences, 18th Ed. (1990, Mack Publishing Co., Easton, Pa.). The pharmaceutical compositions of the disclosure comprise a therapeutically effective amount of the vector encoding the factor (or ETV2 and/or VEGF). These compositions can comprise, in addition to the vector, a pharmaceutically acceptable excipient, carrier, buffer, stabilizer or other materials well known in the art. Such materials should be non-toxic and should not interfere with the efficacy of the active ingredient. The carrier can take a wide variety of forms depending on the form of preparation desired for administration, e.g., intravenous, oral, intramuscular, subcutaneous, intrathecal, epineural or parenteral.

When the vectors of the disclosure are prepared for administration, they may be combined with a pharmaceutically acceptable carrier, diluent or excipient to form a pharmaceutical formulation, or unit dosage form. The total active ingredients in such formulations include from 0.1 to 99.9% by weight of the formulation.

In another aspect of the disclosure, the vectors of the disclosure can be suitably formulated and introduced into the environment of the cell by any means that allows for a sufficient portion of the sample to enter the cell to induce gene silencing, if it is to occur. Many formulations for vectors are known in the art and can be used so long as the vectors gain entry to the target cells so that it can act.

For example, the vectors can be formulated in buffer solutions such as phosphate buffered saline solutions comprising liposomes, micellar structures, and capsids. The pharmaceutical formulations of the vectors of the invention can also take the form of an aqueous or anhydrous solution or dispersion, or alternatively the form of an emulsion or suspension. The pharmaceutical formulations of the vectors of the present invention may include, as optional ingredients, solubilizing or emulsifying agents, and salts of the type that are well-known in the art. Specific non-limiting examples of the carriers and/or diluents that are useful in the pharmaceutical formulations of the present invention include water and physiologically acceptable saline solutions. Other pharmaceutically acceptable carriers for preparing a composition for administration to an individual include, for example, solvents or vehicles such as glycols, glycerol, or injectable organic esters. A pharmaceutically acceptable carrier can contain physiologically acceptable compounds that act, for example, to stabilize or to increase the absorption of the shRNA encoding vector. Other physiologically acceptable carriers include, for example, carbohydrates, such as glucose, sucrose or dextrans, antioxidants, such as ascorbic acid or glutathione, chelating agents, low molecular weight proteins or other stabilizers or excipients, saline, dextrose solutions, fructose solutions, ethanol, or oils of animal, vegetative or synthetic origin. The carrier can also contain other ingredients, for example, preservatives.

It will be recognized that the choice of a pharmaceutically acceptable carrier, including a physiologically acceptable compound, depends, for example, on the route of administration of the composition. The composition containing the vectors can also contain a second reagent such as a diagnostic reagent, nutritional substance, toxin, or additional therapeutic agent. Many agents useful in the treatment of cardiac disease are known in the art and are envisioned for use in conjunction with the vectors of this invention.

Formulations of vectors with cationic lipids can be used to facilitate transfection of the vectors into cells. For example, cationic lipids, such as lipofectin, cationic glycerol derivatives, and polycationic molecules, such as polylysine, can be used. Suitable lipids include, for example, Oligofectamine and Lipofectamine (Life Technologies) which can be used according to the manufacturer's instructions.

Suitable amounts of vector must be introduced and these amounts can be empirically determined using standard methods. Typically, effective concentrations of individual vector species in the environment of a cell will be about 50 nanomolar or less 10 nanomolar or less, or compositions in which concentrations of about 1 nanomolar or less can be used. In other aspects, the methods utilize a concentration of about 200 picomolar or less and even a concentration of about 50 picomolar or less can be used in many circumstances. One of skill in the art can determine the effective concentration for any particular mammalian subject using standard methods.

In cases wherein the transdifferentiation factor(s) is delivered to the heart of an individual in need thereof, the delivery method may be local and may be delivered by any suitable method directly to the heart. The local delivery may be by injection, by stent delivery, or a balloon-based delivery. The local delivery may or may not occur in the heart at a location of cardiac tissue in need, including diseased and/or damaged cardiac tissue. In specific embodiments, the damaged cardiac tissue is damaged from an infarct. The local delivery may be a single delivery, or there may be multiple deliveries over time, such as over the course of 1-7 days, 1-4 weeks, 1-12 months or one or more years.

In cases wherein Gata4 is utilized as a transdifferentiation factor, one example of a Gata4 polynucleotide is at GenBank® Accession No. NM_001308093 (SEQ ID NO:5):

| | |
|---|---|
| 1 | gaccccggct gcggcgagga ggaaggagcc agcctagcag |
| | cttctgcgcc tgtggccgcg |
| 61 | ggtgtcctgg aggcctctcg gtgtgacgag tgggggaccc |
| | gaaggctcgt gcgccacctc |
| 121 | caggcctgga cgctgccctc cgtcttctgc ccccaatagg |
| | tgcgccggac cttcaggccc |
| 181 | tggggtgaat tcagctgctc ctacatcagc ttccggaacc |
| | accaaaaatt caaattggga |
| 241 | ttttccggag taaacaagag cctagagccc tttgctcaat |
| | gctggattta atacgtatat |
| 301 | atttttaagc gagttggttt tttccccttt gatttttgat |
| | cttcgcgaca gttcctccca |
| 361 | cgcatattat cgttgttgcc gtcgttttct ctccccgcgt |
| | ggctccttga cctgcgaggg |
| 421 | agagagagga caccgaagcc gggagctcgc agggaccatg |
| | tatcagagct tggccatggc |
| 481 | cgccaaccac gggccgcccc ccggtgccta cgaggcgggc |
| | ggccccggcg ccttcatgca |
| 541 | cggcgcgggc gccgcgtcct cgccagtcta cgtgcccaca |
| | ccgcgggtgc cctcctccgt |
| 601 | gctgggcctg tcctacctcc agggcggagg cgcgggctct |
| | gcgtccggag gcgcctcggg |
| 661 | cggcagctcc ggtggggccg cgtctggtgc ggggcccggg |
| | acccagcagg gcagcccggg |
| 721 | atggagccag gcgggagccg acggagccgc ttacaccccg |
| | ccgccggtgt cgccgcgctt |
| 781 | ctccttcccg gggaccaccg ggtccctggc ggccgccgcc |
| | gccgctgccg cggcccggga |
| 841 | agctgcggcc tacagcagtg gcggcggagc ggcgggtgcg |
| | ggcctggcgg gccgcgagca |
| 901 | gtacgggcgc gccggcttcg cgggctccta ctccagcccc |
| | tacccggctt acatggccga |
| 961 | cgtgggcgcg tcctgggccg cagccgccgc cgcctccgcc |
| | ggccccttcg acagcccggt |
| 1021 | cctgcacagc ctgcccggcc gggccaaccc ggccgcccga |
| | caccccaatc tcgtagatat |
| 1081 | gtttgacgac ttctcagaag gcagagagtg tgtcaactgt |
| | ggggctatgt ccaccccgct |
| 1141 | ctggaggcga gatgggacgg gtcactatct gtgcaacgcc |
| | tgcggcctct accacaagat |
| 1201 | gaacggcatc aaccggccgc tcatcaagcc tcagcgccgg |
| | ctgtccgcct cccgccgagt |
| 1261 | gggcctctcc tgtgccaact gccagaccac caccaccacg |
| | ctgtggcgcc gcaatgcgga |
| 1321 | gggcgagcct gtgtgcaatg cctgcggcct ctacatgaag |
| | ctccacgggg tccccaggcc |
| 1381 | tcttgcaatg cggaaagagg ggatccaaac cagaaaacgg |
| | aagcccaaga acctgaataa |
| 1441 | atctaagaca ccagcagctc cttcaggcag tgagagcctt |
| | cctcccgcca gcggtgcttc |
| 1501 | cagcaactcc agcaacgcca ccaccagcag cagcgaggag |
| | atgcgtccca tcaagacgga |
| 1561 | gcctggcctg tcatctcact acgggcacag cagctccgtg |
| | tcccagacgt tctcagtcag |
| 1621 | tgcgatgtct ggccatgggc cctccatcca ccctgtcctc |
| | tcggccctga agctctcccc |
| 1681 | acaaggctat gcgtctcccg tcagccagtc tccacagacc |
| | agctccaagc aggactcttg |
| 1741 | gaacagcctg gtcttggccg acagtcacgg ggacataatc |
| | actgcgtaat cttccctctt |
| 1801 | ccctcctcaa attcctgcac ggacctggga cttggaggat |
| | agcaaagaag gaggccctgg |
| 1861 | gctcccaggg gccggcctcc tctgcctggt aatgactcca |
| | gaacaacaac tgggaagaaa |
| 1921 | cttgaagtcg acaatctggt taggggaagc gggtgttgga |
| | ttttctcaga tgcctttaca |
| 1981 | cgctgatggg actggaggga gcccaccctt cagcacgagc |
| | acactgcatc tctcctgtga |
| 2041 | gttggagact tctttcccaa gatgtccttg tcccctgcgt |
| | tccccactgt ggcctagacc |
| 2101 | gtgggttttg cattgtgttt ctagcaccga ggatctgaga |
| | acaagcggag ggccgggccc |
| 2161 | tgggacccct gctccagccc gaatgacggc atctgtttgc |
| | catgtacctg gatgcgacgg |
| 2221 | gcccctgggg acaggccctt gccccatcca tccgcttgag |
| | gcatggcacc gccctgcatc |
| 2281 | cctaatacca aatctgactc caaaattgtg gggtgtgaca |
| | tacaagtgac tgaacacttc |
| 2341 | ctggggagct acaggggcac ttaacccacc acagcacagc |
| | ctcatcaaaa tgcagctggc |
| 2401 | aacttctccc ccaggtgcct tccccctgct gccggccttt |
| | gctccttcac ttccaacatc |
| 2461 | tctcaaaata aaaatccctc ttcccgctct gagcgattca |
| | gctctgcccg cagcttgtac |
| 2521 | atgtctctcc cctggcaaaa caagagctgg gtagtttagc |
| | caaacggcac cccctcgagt |
| 2581 | tcactgcaga cccttcgttc accgtgtcac acatagaggg |
| | gttctgagta agaacaaaac |
| 2641 | gttctgctgc tcaagccagt ctggcaagca ctcagcccag |
| | cctcgaggtc cttctgggga |
| 2701 | gagtgtaagt ggacagagtc ctggtcaggg ggcaggagtg |
| | tcccaagggc tggcccacct |
| 2761 | gctgtctgtc tgctcctcct agcccttggt cagatggcag |
| | ccagagtccc tcaggacctg |
| 2821 | cagcctcgcc ccggcagaag tcttttgtcc aggaggcaaa |
| | aagccagaga ttctgcaaca |
| 2881 | cgaattcgaa gcaaacaaac acaacacaac agaattcctg |
| | gaaagaagac gactgctaag |
| 2941 | acacggcagg ggggcctgga gggagcctcc gactctgagc |
| | tgctccggga tctgccgcgt |
| 3001 | tctcctctgc acattgctgt ttctgcccct gatgctggag |
| | ctcaaggaga ctccttcctc |
| 3061 | tttctcagca gagctgtagc tgactgtggc attactacgc |
| | ctccccacac gcccagaccc |
| 3121 | ctcactccaa aatcctactg gctgtagcag agaatacctt |
| | tgaaccaaga ttctgtttta |
| 3181 | atcatcattt acattgtttt cttccaaagg ccccctcgta |
| | taccctccct aacccacaaa |
| 3241 | cctgttaaca ttgtcttaag gtgaaatggc tggaaaatca |
| | gtatttaact aataaattta |
| 3301 | tctgtattcc tctttcaaaa aa |

In particular embodiments, part or all of SEQ ID NO:5 is utilized in methods of the disclosure. In specific embodiments, a polynucleotide having a specific sequence identity with respect to SEQ ID NO:5 is utilized in methods of the disclosure. In specific cases, a functional fragment of SEQ ID NO:5 is employed, and the term “functional fragment” as used herein refers to a polynucleotide that encodes a polypeptide having the activity of being able to convert endothelial cells or endothelial-like cells to cardiomyocytes alone or in combination with another compound. In specific cases, the fragment has a length of at least about or no more than about 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, or 300 contiguous nucleotides of SEQ ID NO:5. In addition, the fragment may have sequence identity with the corresponding region in SEQ ID NO:5 of 100, 99, 98, 97, 96, 95, 94, 93, 92, 91, 90, 89, 88, 87, 86, 85, 80, 75, or 70% identity. A polynucleotide having certain sequence identity to SEQ ID NO:5 may be used, including 99, 98, 97, 96, 95, 94, 93, 92, 91, 90, 89, 88, 87, 86, 85, 80, 75, or 70% identity to SEQ ID NO:5.

In some examples, a Gata4 polypeptide is delivered to an individual in need thereof, whether it be in the form of being on a vector, associated with a carrier, within a cell (including in a cell on a vector), and so forth. In specific embodiments, the Gata4 polypeptide is a mammalian Gata4 polypeptide, including human, mouse, rat, and so forth. In particular embodiments, one example of a Gata4 polypeptide is at GenBank® Accession No. NP_001295022 (SEQ ID NO:6):

| | |
|---|---|
| 1 | myqslamaan hgpppgayea ggpgafmhga gaasspvyvp |
| | tprvpssvlg lsylqgggag |
| 61 | sasggasggs sggaasgagp gtqqgspgws qagadgaayt |
| | pppvsprfsf pgttgslaaa |
| 121 | aaaaaareaa ayssgggaag aglagreqyg ragfagsyss |
| | pypaymadvg aswaaaaaas |
| 181 | agpfdspvlh slpgranpaa rhpnlvdmfd dfsegrecvn |
| | cgamstplwr rdgtghylcn |
| 241 | acglyhkmng inrplikpqr rlsasrrvgl scancqtttt |
| | tlwrrnaege pvcnacglym |
| 301 | klhgvprpla mrkegiqtrk rkpknlnksk tpaapsgses |
| | 1ppasgassn ssnattssse |
| 361 | emrpiktepg lsshyghsss vsqtfsysam sghgpsihpv |
| | lsalklspqg yaspvsqspq |
| 421 | tsskqdswns lvladshgdi ita |

In particular embodiments, part or all of SEQ ID NO:6 is utilized in methods of the disclosure. In specific embodiments, a polypeptide having a specific sequence identity with respect to SEQ ID NO:6 is utilized in methods of the disclosure. In specific cases, a functional fragment of SEQ ID NO:6 is employed, and the term “functional fragment” as used herein refers to a polypeptide having the activity of being able to convert endothelial cells or endothelial-like cells to cardiomyocytes alone or in combination with another compound. In specific cases, the fragment has a length of at least about or no more than about 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25, or 20 contiguous amino acids of SEQ ID NO:6.

In cases wherein Mef2c is utilized as a transdifferentiation factor, one example of a Mef2c polynucleotide is at GenBank® Accession No. NM_001131005 (SEQ ID NO:7):

| | |
|---|---|
| 1 | aagggggcaa agcctcggtc ttcatagaaa aggagaggag |
| | gcaaacgcag cccaaactgg |
| 61 | ggggtttctc ttcaaagcca gctggtctgg ctttattctg |
| | caggaatttt tttacctgtc |
| 121 | agggtttgga caacaaagcc ctcagcaggt gctgacgggt |
| | acaacttcct ggagaagcag |
| 181 | aaaggcactg gtgccaaaga agagttgcaa actgtgaagt |
| | aacttctatg aagagatgaa |
| 241 | gtaaagaacg gaaggcaaat gattgtggca gtaaagaagt |
| | gtatgtgcag gaacgaatgc |
| 301 | aggaatttgg gaactgagct gtgcaagtgc tgaagaagga |
| | gatttgtttg gaggaaacag |
| 361 | gaaagagaaa gaaaaggaag gaaaaaatac ataatttcag |
| | ggacgagaga gagaagaaaa |
| 421 | acggggacta tggggagaaa aaagattcag attacgagga |
| | ttatggatga acgtaacaga |
| 481 | caggtgacat ttacaaagag gaaatttggg ttgatgaaga |
| | aggcttatga gctgagcgtg |
| 541 | ctgtgtgact gtgagattgc gctgatcatc ttcaacagca |
| | ccaacaagct gttccagtat |
| 601 | gccagcaccg acatggacaa agtgcttctc aagtacacgg |
| | agtacaacga gccgcatgag |
| 661 | agccggacaa actcagacat cgtggaggca ttgaacaaga |
| | aagaaaacaa aggctgtgaa |
| 721 | agccccgatc ccgactcctc ttatgcactc accccacgca |
| | ctgaagaaaa atacaaaaaa |
| 781 | attaatgaag aatttgataa tatgatcaag agtcataaaa |
| | ttcctgctgt tccacctccc |
| 841 | aacttcgaga tgccagtctc catcccagtg tccagccaca |
| | acagtttggt gtacagcaac |
| 901 | cctgtcagct cactgggaaa ccccaaccta ttgccactgg |
| | ctcacccttc tctgcagagg |
| 961 | aatagtatgt ctcctggtgt aacacatcga cctccaagtg |
| | caggtaacac aggtggtctg |
| 1021 | atgggtggag acctcacgtc tggtgcaggc accagtgcag |
| | ggaacgggta tggcaatccc |
| 1081 | cgaaactcac caggtctgct ggtctcacct ggtaacttga |
| | acaagaatat gcaagcaaaa |
| 1141 | tctcctcccc caatgaattt aggaatgaat aaccgtaaac |
| | cagatctccg agttcttatt |
| 1201 | ccaccaggca gcaagaatac gatgccatca gtgaatcaaa |
| | ggataaataa ctcccagtcg |
| 1261 | gctcagtcat tggctacccc agtggtttcc gtagcaactc |
| | ctactttacc aggacaagga |
| 1321 | atgggaggat atccatcagc catttcaaca acatatggta |
| | ccgagtactc tctgagtagt |
| 1381 | gcagacctgt catctctgtc tgggtttaac accgccagcg |
| | ctcttcacct tggttcagta |
| 1441 | actggctggc aacagcaaca cctacataac atgccaccat |
| | ctgccctcag tcagttggga |
| 1501 | gcttgcacta gcactcattt atctcagagt tcaaatctct |
| | ccctgccttc tactcaaagc |
| 1561 | ctcaacatca agtcagaacc tgtttctcct cctagagacc |
| | gtaccaccac cccttcgaga |
| 1621 | tacccacaac acacgcgcca cgaggcgggg agatctcctg |
| | ttgacagctt gagcagctgt |
| 1681 | agcagttcgt acgacgggag cgaccgagag gatcaccgga |
| | acgaattcca ctcccccatt |
| 1741 | ggactcacca gaccttcgcc ggacgaaagg gaaagtccct |
| | cagtcaagcg catgcgactt |
| 1801 | tctgaaggat gggcaacatg atcagattat tacttactag |
| | tttttttttt tttcttgcag |
| 1861 | tgtgtgtgtg tgctatacct taatggggaa ggggggtcga |
| | tatgcattat atgtgccgtg |
| 1921 | tgtggaaaaa aaaaaagtca ggtactctgt tttgtaaaag |
| | tacttttaaa ttgcctcagt |
| 1981 | gatacagtat aaagataaac agaaatgctg agataagctt |
| | agcacttgag ttgtacaaca |
| 2041 | gaacacttgt acaaaataga ttttaaggct aacttctttt |
| | cactgttgtg ctcctttgca |
| 2101 | aaatgtatgt tacaatagat agtgtcatgt tgcaggttca |
| | acgttattta catgtaaata |
| 2161 | gacaaaagga aacatttgcc aaaagcggca gatctttact |
| | gaaagagaga gcagctgtta |
| 2221 | tgcaacatat agaaaaatgt atagatgctt ggacagaccc |
| | ggtaatgggt ggccattggt |
| 2281 | aaatgttagg aacacaccag gtcacctgac atcccaagaa |
| | tgctcacaaa cctgcaggca |
| 2341 | tatcattggc gtatggcact cattaaaaag gatcagagac |
| | cattaaaaga ggaccatacc |
| 2401 | tattaaaaaa aaatgtggag ttggagggct aacatattta |
| | attaaataaa taaataaatc |
| 2461 | tgggtctgca tctcttatta aataaaaata taaaaatatg |
| | tacattacat tttgcttatt |
| 2521 | ttcatataaa aggtaagaca gagtttgcaa agcatttgtg |
| | gctttttgta gtttacttaa |
| 2581 | gccaaaatgt gtttttttcc ccttgatagc ttcgctaata |
| | ttttaaacag tcctgtaaaa |
| 2641 | aaccaaaaag gactttttgt atagaaagca ctaccctaag |
| | ccatgaagaa ctccatgctt |
| 2701 | tgctaaccaa gataactgtt ttctctttgt agaagttttg |
| | tttttgaaat gtgtatttct |
| 2761 | aattatataa aatattaaga atcttttaaa aaaatctgtg |
| | aaattaacat gcttgtgtat |
| 2821 | agctttctaa tatatataat attatggtaa tagcagaagt |
| | tttgttatct taatagcggg |
| 2881 | aggggggtat atttgtgcag ttgcacattt gagtaactat |
| | tttctttctg ttttctttta |
| 2941 | ctctgcttac attttataag tttaaggtca gctgtcaaaa |
| | ggataacctg tggggttaga |
| 3001 | acatatcaca ttgcaacacc ctaaattgtt tttaatacat |
| | tagcaatcta ttgggtcaac |
| 3061 | tgacatccat tgtatatact agtttctttc atgctatttt |
| | tattttgttt tttgcatttt |
| 3121 | tatcaaatgc agggcccctt tctgatctca ccatttcacc |
| | atgcatcttg gaattcagta |
| 3181 | agtgcatatc ctaacttgcc catattctaa atcatctggt |
| | tggttttcag cctagaattt |
| 3241 | gatacgcttt ttagaaatat gcccagaata gaaaagctat |
| | gttggggcac atgtcctgca |
| 3301 | aatatggccc tagaaacaag tgatatggaa tttacttggt |
| | gaataagtta taaattccca |
| 3361 | cagaagaaaa atgtgaaaga ctgggtgcta gacaagaagg |
| | aagcaggtaa agggatagtt |
| 3421 | gctttgtcat ccgtttttaa ttattttaac tgacccttga |
| | caatcttgtc agcaatatag |
| 3481 | gactgttgaa caatcccggt gtgtcaggac ccccaaatgt |
| | cacttctgca taaagcatgt |
| 3541 | atgtcatcta ttttttcttc aataaagaga tttaatagcc |
| | atttcaagaa atcccataaa |
| 3601 | gaacctctct atgtcccttt ttttaattta aaaaaaatga |
| | ctcttgtcta atattcgtct |
| 3661 | ataagggatt aattttcaga ccctttaata agtgagtgcc |
| | ataagaaagt caatatatat |
| 3721 | tgtttaaaag atatttcagt ctaggaaaga ttttccttct |
| | cttggaatgt gaagatctgt |
| 3781 | cgattcatct ccaatcatat gcattgacat acacagcaaa |
| | gaagatatag gcagtaatat |
| 3841 | caacactgct atatcatgtg taggacattt cttatccatt |
| | ttttctcttt tacttgcata |
| 3901 | gttgctatgt gtttctcatt gtaaaaggct gccgctgggt |
| | ggcagaagcc aagagacctt |
| 3961 | attaactagg ctatattttt cttaacttga tctgaaatcc |
| | acaattagac cacaatgcac |
| 4021 | ctttggttgt atccataaag gatgctagcc tgccttgtac |
| | taatgtttta tatattaaaa |
| 4081 | aaaaaaaatc tatcaaccat ttcatatata tcccactact |
| | caaggtatcc atggaacatg |
| 4141 | aaagaataac atttatgcag aggaaaaaca aaaacatccc |
| | tgaaaatata cacactcata |
| 4201 | cacacacacg cacaggggaa taaaataaga aaatcatttt |
| | cctcaccata gacttgatcc |
| 4261 | catccttaca acccatcctt ctaacttgat gtgtataaaa |
| | tatgcaaaca tttcacaaat |
| 4321 | gttctttgtc atttcaaaat actttagtat atcaatatca |
| | gtagatacca gtgggtggga |
| 4381 | aagggtcatt acatgaaaat atgaagaaat agccatatta |
| | gttttttaac ctgcaatttg |
| 4441 | cctcagcaac aaagaaaaag tgaattttta atgctgaaga |
| | taaagtaagc taaagtacca |
| 4501 | gcagaagcct tggctattta tagcagttct gacaatagtt |
| | ttataagaac atgaagagaa |
| 4561 | cagaatcact tgaaaatgga tgccagtcat ctcttgttcc |
| | cactactgaa ttcttataaa |
| 4621 | gtggtggcaa gatagggaag ggataatctg agaattttta |
| | aaagatgatt taatgagaag |
| 4681 | aagcacaatt ttgattttga tgagtcactt tctgtaaaca |
| | atcttggtct atctttaccc |
| 4741 | ttatacctta tctgtaattt accatttatt gtatttgcaa |
| | agctagtatg gtttttaatc |
| 4801 | acagtaaatc ctttgtattc cagactttag ggcagagccc |
| | tgagggagta ttattttaca |
| 4861 | taacccgtcc tagagtaaca ttttaggcaa cattcttcat |
| | tgcaagtaaa agatccataa |
| 4921 | gtggcatttt acacggctgc gagtattgtt atatctaatc |
| | ctattttaaa agatttttgg |
| 4981 | taatatgaag cttgaatact ggtaacagtg atgcaatata |
| | cgcaagctgc acaacctgta |
| 5041 | tattgtatgc attgctgcgt ggaggctgtt tatttcaacc |
| | tttttaaaaa ttgtgttttt |
| 5101 | tagtaaaatg gcttattttt tcccaaaggt ggaatttagc |
| | attttgtaat gatgaatata |
| 5161 | aaaatacctg tcatccccag atcatttaaa agttaactaa |
| | agtgagaatg aaaaaacaaa |
| 5221 | attccaagac actttttaaa agaatgtctg ccctcacaca |
| | cttttatgga tttgtttttc |
| 5281 | ttacataccc atcttttaac ttagagatag cattttttgc |
| | cctctttatt ttgttgtttg |
| 5341 | tttctccaga gagtaaacgc tttgtagttc tttctttaaa |
| | aaacattttt tttaaagaag |
| 5401 | aagaagccac ttgaaccctc aataaaggct gttgcctaag |
| | catggcatac ttcatctgtt |
| 5461 | ctcatttgtg ccatctgccg tgatgtcgtc acttttatgg |
| | cgttaatttc ctgccactac |
| 5521 | agatcttttg aagattgctg gaatactggt gtctgttaga |
| | atgcttcaga ctacagatgt |
| 5581 | aattaaaggc ttttcttaat atgttttaac caaagatgtg |
| | gagcaatcca agccacatat |
| 5641 | cttctacatc aaatttttcc attttggtta ttttcataat |
| | ctggtattgc attttgcctt |
| 5701 | ccctgttca acctcaaatt gattcatacc tcagtttaat |
| | tcagagaggt cagttaagtg |
| 5761 | acggattctg ttgtggtttg aatgcagtac cagtgttctc |
| | ttcgagcaaa gtagacctgg |
| 5821 | gtcactgtag gcataggact tggattgctt cagatggttt |
| | gctgtatcat ttttcttctt |
| 5881 | tttcttttcc tggggacttg tttccattaa atgagagtaa |
| | ttaaaatcgc ttgtaaatga |
| 5941 | gggcatacaa gcatttgcaa caaatattca aatagaggct |
| | cacagcggca taagctggac |
| 6001 | tttgtcgcca ctagatgaca agatgttata actaagttaa |
| | accacatctg tgtatctcaa |
| 6061 | gggacttaat tcagctgtct gtagtgaata aaagtgggaa |
| | attttcaaaa gtttctcctg |
| 6121 | ctggaaataa ggtataattt gtattttgca gacaattcag |
| | taaagttact ggctttctta |
| 6181 | gtgaaaaaaa aaaa |

In particular embodiments, part or all of SEQ ID NO:7 is utilized in methods of the disclosure. In specific embodiments, a polynucleotide having a specific sequence identity with respect to SEQ ID NO:7 is utilized in methods of the disclosure. In specific cases, a functional fragment of SEQ ID NO:7 is employed, and the term “functional fragment” as used herein refers to a polynucleotide that encodes a polypeptide having the activity of being able to convert endothelial cells or endothelial-like cells to cardiomyocytes alone or in combination with another compound. In specific cases, the fragment has a length of at least about or no more than about 6000, 5900, 5800, 5700, 5600, 5500, 5400, 5300, 5200, 5100, 5000, 4900, 4800, 4700, 4600, 4500, 4400, 4300, 4200, 4100, 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, or 300 contiguous nucleotides of SEQ ID NO:7. In addition, the fragment may have sequence identity with the corresponding region in SEQ ID NO:7 of 100, 99, 98, 97, 96, 95, 94, 93, 92, 91, 90, 89, 88, 87, 86, 85, 80, 75, or 70% identity. A polynucleotide having certain sequence identity to SEQ ID NO:7 may be used, including 99, 98, 97, 96, 95, 94, 93, 92, 91, 90, 89, 88, 87, 86, 85, 80, 75, or 70% identity to SEQ ID NO:7.

In some examples, a Mef2c polypeptide is delivered to an individual in need thereof, whether it be in the form of being on a vector, associated with a carrier, within a cell (including in a cell on a vector), and so forth. In specific embodiments, the Mef2c polypeptide is a mammalian Mef2c polypeptide, including human, mouse, rat, and so forth. In particular embodiments, one example of a Mef2c polypeptide is at GenBank® Accession No. NP_001124477 (SEQ ID NO:8):

| | |
|---|---|
| 1 | mgrkkiqitr imdernrqvt ftkrkfglmk kayelsvlcd |
| | ceialiifns tnklfqyast |
| 61 | dmdkvllkyt eynephesrt nsdivealnk kenkgcespd |
| | pdssyaltpr teekykkine |
| 121 | efdnmikshk ipavpppnfe mpvsipvssh nslvysnpvs |
| | slgnpnllpl ahpslqrnsm |
| 181 | spgvthrpps agntgglmgg dltsgagtsa gngygnprns |
| | pgllvspgnl nknmqakspp |
| 241 | pmnlgmnnrk pdlrvlippg skntmpsvnq rinnsqsaqs |
| | latpvvsvat ptlpgqgmgg |
| 301 | ypsaisttyg teyslssadl sslsgfntas alhlgsvtgw |
| | qqqhlhnmpp salsqlgact |
| 361 | sthlsqssnl slpstqslni ksepvspprd rtttpsrypq |
| | htrheagrsp vdslsscsss |
| 421 | ydgsdredhr nefhspiglt rpspderesp svkrmrlseg |
| | wat |

In particular embodiments, part or all of SEQ ID NO:8 is utilized in methods of the disclosure. In specific embodiments, a polypeptide having a specific sequence identity with respect to SEQ ID NO:8 is utilized in methods of the disclosure. In specific cases, a functional fragment of SEQ ID NO:8 is employed, and the term “functional fragment” as used herein refers to a polypeptide having the activity of being able to convert endothelial cells or endothelial-like cells to cardiomyocytes alone or in combination with another compound. In specific cases, the fragment has a length of at least about or no more than about 410, 400, 390, 380, 370, 360, 350, 340, 330, 320, 310, 300, 290, 280, 270, 260, 250, 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25, or 20 contiguous amino acids of SEQ ID NO:8.

In cases wherein Tbx5 is utilized as a transdifferentiation factor, one example of a Tbx5 polynucleotide is at GenBank® Accession No. Y09445 (SEQ ID NO:9):

| | |
|---|---|
| 1 | catgccttat gcaagagacc tcagtccccc ggaacaactc |
| | gatttccttc caatagaggt |
| 61 | ctgaggtgga ctcccacctc ccttcgtgaa gagttccctc |
| | ctctccccct tcctaagaaa |
| 121 | gtcgatcttg gctctatttg tgtcttatgt tcatcaccct |
| | cattcctccg gagaaagccg |
| 181 | ggttggttta tgtctttatt tattcccggg gccaagacgt |
| | ccggaacctg tggctgcgca |
| 241 | gacccggcac tgataggcga agacggagag aaatttacct |
| | cccgccgctg ccccccagcc |
| 301 | aaacgtgaca gcgcgcgggc cggttgcgtg actcgtgacg |
| | tctccaagtc ctataggtgc |
| 361 | agcggctggt gagatagtcg ctatcgcctg gttgcctctt |
| | tattttactg gggtatgcct |
| 421 | ggtaataaac agtaatattt aatttgtcgg agaccacaaa |
| | ccaaccttga gctgggaggt |
| 481 | acgtgctctt cttgacagac gttggaagaa gacctggcct |
| | aaagaggtct cttttggtgg |
| 541 | tccttttcaa agtcttcacc tgagccctgc tctccagcga |
| | ggcgcactcc tggcttttgc |
| 601 | gctccaaaga agaggtggga tagttggaga gcagaacctt |
| | gcgcgggcac aggcctgggc |
| 661 | gcaccatggc cgacgcagac gaggctttgg ctggcgcaca |
| | cctctggagc ctgacgcaaa |
| 721 | agacctgcct gcgattcgaa ccgagagcgc gctcggggcc |
| | cccagcaagt ccccccggtc |
| 781 | gtccccgcag ccgccttcac ccagcaggca tggagggaat |
| | caaagtgttt ctccatgaaa |
| 841 | gagaactgtg gctaaaattc cacgaagtca cggaaatgat |
| | cataaccaag gctggaaggc |
| 901 | ggatgtttcc cagttacaaa gtgaaggtga cgggcattaa |
| | tcccaaaacg aagtacattc |
| 961 | ttctcatgga cattgtacct gcggacgatc acagatacaa |
| | attcgcagat aataaatggt |
| 1021 | gtgtgacggg caaagctgag cccgccatgg ctggccgcct |
| | gtacgtgcac ccagactccc |
| 1081 | ccgccaccgg ggcgcattgg atgaggcagc tcgtctcctt |
| | ccagaaactc aagctcacca |
| 1141 | acaaccacct ggacccattt gggcatatta ttctaaattc |
| | catgcacaaa taccagccta |
| 1201 | gattacacat cgtgaaagcg gatgaaaata atggatttgg |
| | ctcaaaaaat acagcgttct |
| 1261 | gcactcacgt ctttcctgag actgcgttta tagcagtgac |
| | ttcctaccag aaccacaaga |
| 1321 | tcacgcaatt aaagattgag aataatccct ttgccaaagg |
| | atttcggggc agtgatgaca |
| 1381 | tggagctgca cagaatgtca agaatgcaaa gtaaagaata |
| | tcccgtggtc cccaggagca |
| 1441 | ccgtgaggca aaaagtggcc tccaaccaca gtcctttcag |
| | cagcgagtct cgagctctct |
| 1501 | ccacctcatc caatttgggg tcccaatacc agtgtgagaa |
| | tggtgtttcc ggcccctccc |
| 1561 | aggacctcct gcctccaccc aacccatacc cactgcccca |
| | ggagcatagc caaatttacc |
| 1621 | attgtaccaa gaggaaagag gaagaatgtt ccaccacaga |
| | ccatccctat aagaagccct |
| 1681 | acatggagac atcacccagt gaagaagatt ccttctaccg |
| | ctctagctat ccacagcagc |
| 1741 | agggcctggg tgcctcctac aggacagagt cggcacagcg |
| | gcaagcttgc atgtatgcca |
| 1801 | gctctgcgcc ccccagcgag cctgtgccca gcctagagga |
| | catcagctgc aacacgtggc |
| 1861 | caagcatgcc ttcctacagc agctgcaccg tcaccaccgt |
| | gcagccatgg acaggctacc |
| 1921 | ctaccagcac ttctccgctc acttcacctc ggggcccctg |
| | gtccctcggc tggctggcat |
| 1981 | ggcaaccatg gctccccaca gctgggagag ggaatgttcc |
| | cagcaccaga cctcccgtgg |
| 2041 | cccaccagcc tgtggtcagc agtgtggggc cccaaactgg |
| | cctgcagtcc cctggcaccc |
| 2101 | ttcagccccc tgagttcctc tactctcatg gcgtgcaagg |
| | actctatccc ctcatcagta |
| 2161 | ccactctgtg cacggagttg gcatggtgca gagtggagcg |
| | acaatagcta aagtgaggcc |
| 2221 | tgcttcacaa cagacatttc ctagagaaag agagagagag |
| | aggagaaaga gagagaagga |
| 2281 | gagagacagt agccaagaga accccacaga caagattttt |
| | catttcaccc aatgttcaca |
| 2341 | tctgcactca aggtcgctgg atgctgatct aatcagtagc |
| | ttgaaaccac aattttaaaa |
| 2401 | atgtgacttt cttgttttgt ctcaaaactt aaaaaaaaaa a |

In particular embodiments, part or all of SEQ ID NO:9 is utilized in methods of the disclosure. In specific embodiments, a polynucleotide having a specific sequence identity with respect to SEQ ID NO:9 is utilized in methods of the disclosure. In specific cases, a functional fragment of SEQ ID NO:9 is employed, and the term “functional fragment” as used herein refers to a polynucleotide that encodes a polypeptide having the activity of being able to convert endothelial cells or endothelial-like cells to cardiomyocytes alone or in combination with another compound. In specific cases, the fragment has a length of at least about or no more than about 6000, 5900, 5800, 5700, 5600, 5500, 5400, 5300, 5200, 5100, 5000, 4900, 4800, 4700, 4600, 4500, 4400, 4300, 4200, 4100, 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, or 300 contiguous nucleotides of SEQ ID NO:9. In addition, the fragment may have sequence identity with the corresponding region in SEQ ID NO:9 of 100, 99, 98, 97, 96, 95, 94, 93, 92, 91, 90, 89, 88, 87, 86, 85, 80, 75, or 70% identity. A polynucleotide having certain sequence identity to SEQ ID NO:9 may be used, including 99, 98, 97, 96, 95, 94, 93, 92, 91, 90, 89, 88, 87, 86, 85, 80, 75, or 70% identity to SEQ ID NO:9.

In some examples, a Tbx5 polypeptide is delivered to an individual in need thereof, whether it be in the form of being on a vector, associated with a carrier, within a cell (including in a cell on a vector), and so forth. In specific embodiments, the Tbx5 polypeptide is a mammalian Tbx5 polypeptide, including human, mouse, rat, and so forth. In particular embodiments, one example of a Tbx5 polypeptide is at GenBank® Accession No. CAA70592 (SEQ ID NO:10):

| | |
|---|---|
| 1 | madadealag ahlwsltqkt clrfeprars gppasppgrp |
| | rsrlhpagme gikvflhere |
| 61 | lwlkfhevte miitkagrrm fpsykvkvtg inpktkyill |
| | mdivpaddhr ykfadnkwcv |
| 121 | tgkaepamag rlyvhpdspa tgahwmrqlv sfqklkltnn |
| | hldpfghiil nsmhkyqprl |
| 181 | hivkadenng fgskntafct hvfpetafia vtsyqnhkit |
| | qlkiennpfa kgfrgsddme |
| 241 | lhrmsrmqsk eypvvprstv rqkvasnhsp fssesralst |
| | ssnlgsqyqc engvsgpsqd |
| 301 | llpppnpypl pqehsqiyhc tkrkeeecst tdhpykkpym |
| | etspseedsf yrssypqqqg |
| 361 | lgasyrtesa qrqacmyass appsepvpsl ediscntwps |
| | mpsyssctvt tvqpwtgypt |
| 421 | stspltsprg pwslgwlawq pwlptagrgn vpstrppvah |
| | qpvvssvgpq tglqspgtlq |
| 481 | ppeflyshgv qglyplistt lctelawcry erq |

In particular embodiments, part or all of SEQ ID NO:10 is utilized in methods of the disclosure. In specific embodiments, a polypeptide having a specific sequence identity with respect to SEQ ID NO:10 is utilized in methods of the disclosure. In specific cases, a functional fragment of SEQ ID NO:10 is employed, and the term “functional fragment” as used herein refers to a polypeptide having the activity of being able to convert endothelial cells or endothelial-like cells to cardiomyocytes alone or in combination with another compound. In specific cases, the fragment has a length of at least about or no more than about 410, 400, 390, 380, 370, 360, 350, 340, 330, 320, 310, 300, 290, 280, 270, 260, 250, 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25, or 20 contiguous amino acids of SEQ ID NO:10.

In certain embodiments, following delivery of an effective amount of the one or more transdifferentiation factors to the heart of an individual, there may or may not be assessment whether or not cardiomyocytes are being generated. Cardiac tissue from the individual may be assayed for one or more particular markers of cardiomyocyte cells (for example, cardiac troponin C). In some cases, the individual may be monitored by standard means to identify if there is improvement of cardiac tissue following delivery of the one or more transdifferentiation factors. For example, the individual may be subjected to ultrasound, a stress test, an electrocardiogram, MRI, PET, echocardiogram, or a combination thereof.

In specific embodiments, cells utilized in methods of the disclosure employ regulatable expression of exogenous gene products (e.g., using reverse tetracycline-controlled transactivator [rtTA] or other regulatable promoters; Cre-mediated gene expression).

### IV. Therapeutic Applications of the Differentiated Cells

Methods of the disclosure may be utilized in an individual in need of cell therapy. In particular embodiments, an effective amount of differentiated cells produced by methods encompassed herein are provided to an individual in need thereof. For example, for cardiomyocyte embodiments, individuals receiving methods and compositions of the disclosure include those having had or susceptible to or suspected of having cardiac disease, including ischemic disease or myocardial infarction. In an individual having had a myocardial infarction, methods of the disclosure encompass in specific aspects the conversion of endogenous scar fibroblasts in areas of the myocardial infarction into the cardiomyocytes, thereby regenerating contractile myocardial tissue from infarcted myocardium.

When providing methods and compositions of the disclosure to an individual that has had a myocardial infarction, for example, the timing of the delivery may be within a specific time period following the infarct. In specific embodiments, the individual is provided the disclosed therapy within 1-60 minutes, 1-24 hours, 1-7 days, 1-4 weeks, 1-12 months, or one or more years of the infarct. In specific embodiments, when referring to the timing of the therapy, the reference is to the ETV2 and/or VEGF fibroblast/endothelial cell production or the transdifferentiation factor/cardiomyocyte steps. In specific embodiments, the delivery occurs during a chronic, established infarction.

Embodiments of the present disclosure are directed to methods and/or compositions related to therapy and/or prevention of one or more cardiac-related medical conditions. Embodiments of the present disclosure concern regeneration of tissue, including muscle tissue, such as myocardial tissue, through the reprogramming of existing cells in the heart that are not cardiomyocytes. Certain embodiments relate to reversal of a cardiac medical condition (or improvement of at least one symptom thereof), including at least cardiac disease, cardiomyopathy, cardiotoxicity, congestive heart failure, ischemic heart disease, myocardial infarction, coronary artery disease, cor pulmonale, inflammatory heart disease; inflammatory cardiomegaly; myocarditis; congenital heart disease; rheumatic heart disease, cardiac systolic dysfunction, cardiac diastolic dysfunction, angina, dilated cardiomyopathy, idiopathic cardiomyopathy, or other conditions resulting in cardiac fibrosis, for example.

In particular aspects of the disclosure, cardiomyopathy is the cardiac medical condition to be treated. The cardiac medical condition (including, for example, cardiomyopathy) may be caused by one or more of a variety of characteristics, including, for example, long-term high blood pressure; heart valve problems; heart tissue damage (such as from one or more previous heart attack(s) or chronic or acute and/or recurrent episodes or sequelae of ischemic heart disease); chronic rapid heart rate; metabolic disorders, such as thyroid disease or diabetes; nutritional deficiencies of essential vitamins or minerals, such as thiamin (vitamin B-1), selenium, calcium and/or magnesium; pregnancy; alcohol abuse; drug abuse, including of narcotics or prescription drugs, such as cocaine or antidepressant medications, such as tricyclic antidepressants; use of some chemotherapy drugs to treat cancer (including Adriamycin); certain viral infections; hemochromatosis and/or an unknown cause or undetected cause, i.e. idiopathic cardiomyopathy.

In some cases, methods and compositions of the present disclosure are employed for treatment or prevention of one or more cardiac medical conditions or delay of onset of one or more cardiac medical conditions or reduction of extent of one or more symptoms of one or more cardiac medical conditions. In particular cases, such prevention, delay or onset, or reduction of extent of one or more symptoms, occurs in an individual that is at risk for a cardiac medical condition. Exemplary risk factors include one or more of the following: age, gender (male, although it occurs in females), high blood pressure, high serum cholesterol levels, tobacco smoking, excessive alcohol consumption, sugar consumption, family or personal history, obesity, lack of physical activity, psychosocial factors, diabetes mellitus, overweight, genetic predisposition, and/or exposure to air pollution.

Embodiments of the disclosure include delivery of one or more polynucleotides (which may also be referred to as nucleic acids) or polypeptides produced therefrom that stimulate transdifferentiation or direct reprogramming of cells (such as muscle cells, including cardiomyocytes) and/or tissue (including cardiac tissue). Particular aspects for such embodiments result in reversal of one or more cardiac medical conditions. Certain aspects for such embodiments result in improvement of at least one symptom of a cardiac medical condition. In exemplary embodiments, the cardiac medical condition is heart failure. The heart failure may be the result of one or more causes, including coronary artery disease and heart attack, high blood pressure, faulty heart valves, cardiomyopathy (such as caused by disease, infection, alcohol abuse and the toxic effect of drugs, such as cocaine or some drugs used for chemotherapy), idiopathic cardiomyopathy and/or genetic factors.

Particular but exemplary indications of embodiments of the disclosure include at least applications for 1) heart failure, including congestive heart failure; 2) prevention of ventricular remodeling; and/or 3) cardiomyopathy. Other indications may also include coronary artery disease, ischemic heart disease, valvular heart disease, etc. In specific embodiments, methods and compositions of the disclosure provide cardiomyocyte regeneration that is sufficient to reverse established cardiomyopathy, congestive heart failure, and prevention of ventricular remodeling.

In cases where the individual has cardiomyopathy, the cardiomyopathy may be ischemic or non-ischemic cardiomyopathy. The cardiomyopathy may be caused by long-term high blood pressure, heart valve problems, heart tissue damage from a previous heart attack, chronic rapid heart rate, metabolic disorders, nutritional deficiencies, pregnancy, alcohol abuse, drug abuse, chemotherapy drugs, viral infection, hemochromatosis, genetic condition, elevated cholesterol levels, or a combination thereof. Cardiomyopathy may also have no identified cause, i.e. idiopathic cardiomyopathy.

Embodiments of the disclosure include methods and/or compositions for regeneration of cardiac muscle and reversal of myocardial ischemic injury, for example. In particular embodiments, there are methods for reprogramming of cardiac scar cells (fibroblasts) into adult cardiac muscle cells in mammalian hearts in an individual that has had a cardiac medical condition, such as acute or chronic ischemic injury, for example.

In specific embodiments, any cardiac method encompassed by the disclosure comprises the step of delivering to the individual with or susceptible to a cardiac condition an additional cardiac therapy, such as one that comprises drug therapy, surgery, ventricular assist device (VAD) implantation, video assisted thoracotomy (VAT) coronary bypass, percutaneous coronary intervention (PCI), intra-aortic balloon pump (IABP), extracorporeal membrane oxygenation (ECMO), or a combination thereof.

In cases wherein the methods of the disclosure produce neural cells, including neurons utilizing one or more transdifferentiation factors, the individual may be in need of such cells because they have a neural disease of the brain, spine, or nerves. Examples include ALS; Arteriovenous Malformation; Brain Aneurysm; Brain Tumors; Dural Arteriovenous Fistulae; Epilepsy; Headache; Memory Disorders; Multiple Sclerosis; Parkinson's disease; Peripheral Neuropathy; Post-Herpetic Neuralgia; Spinal Cord Tumor; Stroke, or a combination thereof.

In cases wherein the methods of the disclosure produce hepatocytes utilizing one or more transdifferentiation factors, the individual may be in need of such cells because they have a liver disease, such as Alagille Syndrome; Alcohol-Related Liver Disease; Alpha-1 Antitrypsin Deficiency; Autoimmune Hepatitis; Benign Liver Tumors; Biliary Atresia; Cirrhosis; Crigler-Najjar Syndrome; Galactosemia; Gilbert Syndrome; Hemochromatosis; Hepatitis A; Hepatitis B; Hepatitis C; Hepatic Encephalopathy; Intrahepatic Cholestasis of Pregnancy (ICP); Lysosomal Acid Lipase Deficiency (LAL-D); Liver Cysts; Liver Cancer; Newborn Jaundice; Non-Alcoholic Fatty Liver Disease; Primary Biliary Cholangitis (PBC); Primary Sclerosing Cholangitis (PSC); Reye Syndrome; Type I Glycogen Storage Disease; Wilson Disease, or a combination thereof.

In cases wherein the methods of the disclosure produce skeletal myocytes utilizing one or more transdifferentiation factors, the individual may be in need of such cells because they have a muscle disease, such as Duchenne muscular dystrophy (DMD) or Becker muscular dystrophy (BMD), or a combination thereof.

In cases wherein the methods of the disclosure produce chondrocytes utilizing one or more transdifferentiation factors, the individual may be in need of such cells because they have a cartilage or joint disease or injury, such as degenerative disc, polychondritis, osteoarthritis, or a combination thereof.

In cases wherein the methods of the disclosure produce pancreatic beta cells utilizing one or more transdifferentiation factors, the individual may be in need of such cells because they have pancreatitis or pancreatic cancer, or a combination thereof.

In cases wherein the methods of the disclosure produce adipocytes utilizing one or more transdifferentiation factors, the individual may be in need of such cells because they have wasting syndrome, HIV, cancer, cachexia, anorexia, unexplained weight loss, or a combination thereof.

In cases wherein the methods of the disclosure produce osteoblasts utilizing one or more transdifferentiation factors, the individual may be in need of such cells because they have bone fracture or breakage or injury of any kind, bone cancer, osteogenesis imperfecta, osteomyelitis, osteoporosis, hip dysplasia, or a combination thereof.

### V. Kits of the Disclosure

Any of the compositions described herein may be comprised in a kit. In a non-limiting example, ETV2 and/or VEGF and one or more transdifferentiation factors may be comprised in a kit. The kit may additionally comprise additional agents for diagnosis and/or therapy of a medical condition, for example a cardiac condition.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the one or more compositions in close confinement for commercial sale. Such containers may include injection or blow-molded plastic containers into which the desired vials are retained.

The composition may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

The kits of the present disclosure will also typically include a means for containing the vials in close confinement for commercial sale, such as, e.g., injection and/or blow-molded plastic containers into which the desired vials are retained.

In particular embodiments, the kit comprises reagents and/or tools for determining that an individual has a particular medical condition, such as a cardiac medical condition. In some embodiments, the kit comprises one or more additional therapies for a cardiac-related medical condition, such as one or more of ACE Inhibitor, aldosterone inhibitor, angiotensin II receptor blocker (ARBs); beta-blocker, calcium channel blocker, cholesterol-lowering drug, digoxin, diuretics, inotropic therapy, potassium, magnesium, vasodilator, anticoagulant medication, aspirin, TGF-beta inhibitor, and a combination thereof. In specific embodiments, an individual receives angiogenic therapy before, during, or after the therapy of the present disclosure. Examples of angiogenic therapies include fibroblast growth factor (FGF); vascular endothelial growth factor (VEGF); angiopoietins, Ang1 and Ang2; matrix metalloproteinase (MMP); Delta-like ligand 4 (DII4); or peptides thereof; or combinations thereof.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Direct Reprogramming of Cardiac Fibroblasts into Cardiomyocytes Using an Endothelial Cell Transdifferentiation Strategy

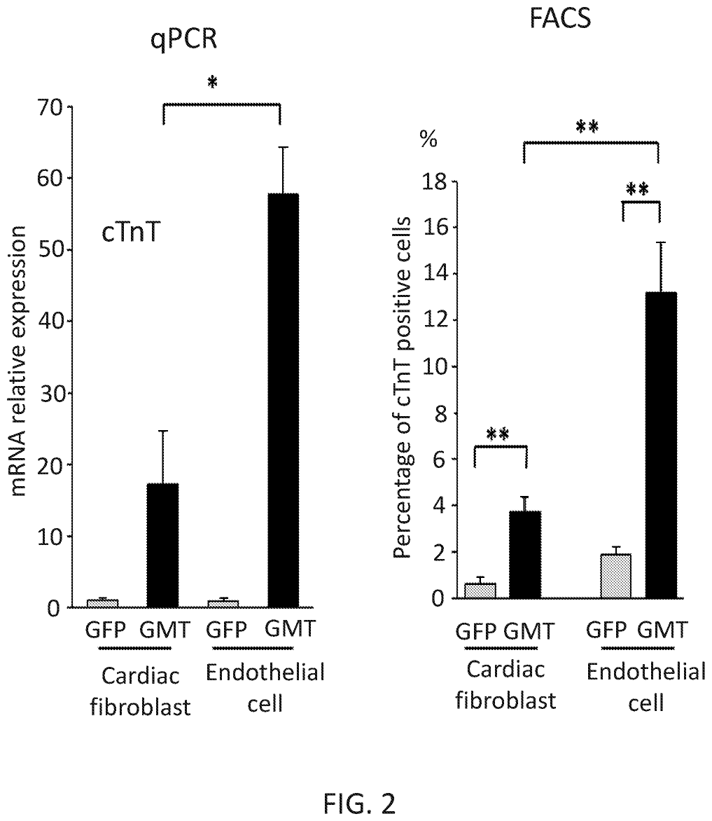

FIG. 2 shows cardiac troponin T expression levels as a measurement of cardiomyocyte production when endothelial cells are exposed to GMT compared to when fibroblasts are exposed to GMT. Endothelial cells are reprogrammed by GMT with higher efficiency than fibroblasts. *: p<0.05; **: p<0.01.

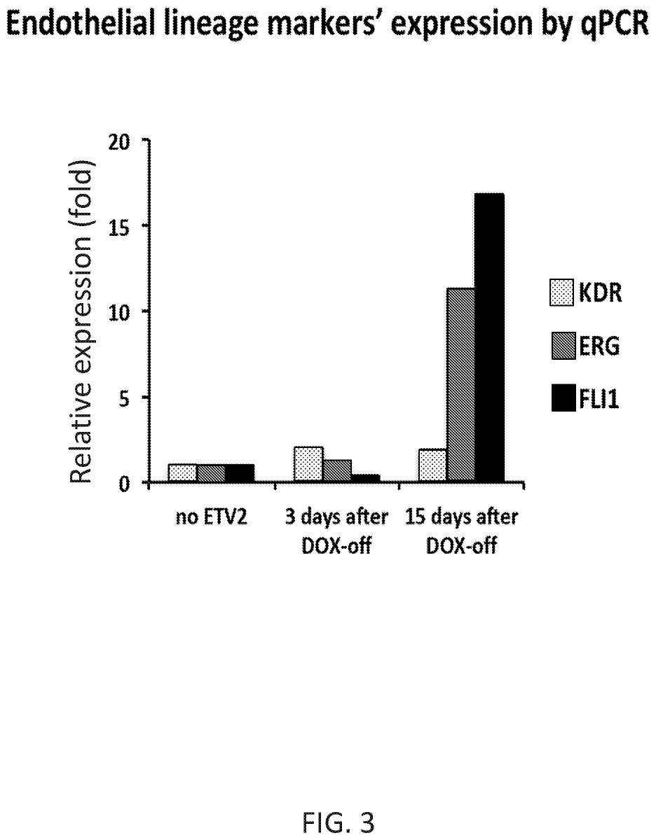

ETV2 administration enhanced endothelial-like cell differentiation of fibroblasts, as shown in FIG. 3 of the outcome of cardiac fibroblasts infected with ETV2 for 10 days. These cells were harvested 3 days or 15 days after DOX was stopped. In particular, FIG. 3 shows that endothelial lineage markers, KDR, ERG, and FLI1 were up-regulated in ETV-infected cells. Data is shown as relative fold to no ETV2 group.

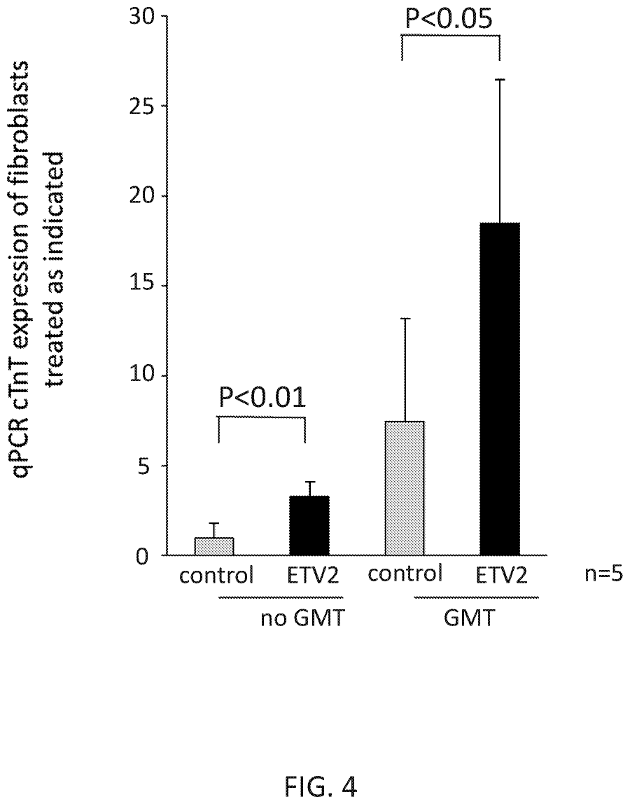

FIG. 4 demonstrates that cardiac fibroblasts infected with lentivirus encoding ETV2 and GMT demonstrate significantly greater cTnT expression than cells not infected with both ETV2 and GMT; Group 1 (left pair) without GMT administration and Group 2 (right pair) with GMT administration. Each group has sub-groups, with or without ETV2. ETV2 was administered 10 days prior to GMT administration. Fourteen days after GMT administration, cTnT expression was analyzed by qPCR. In Group 2, expression of the iCM marker cTnT was significantly greater than that demonstrated by cells receiving GMT alone. Data is shown as fold change relative to no ETV2 and no GMT group.

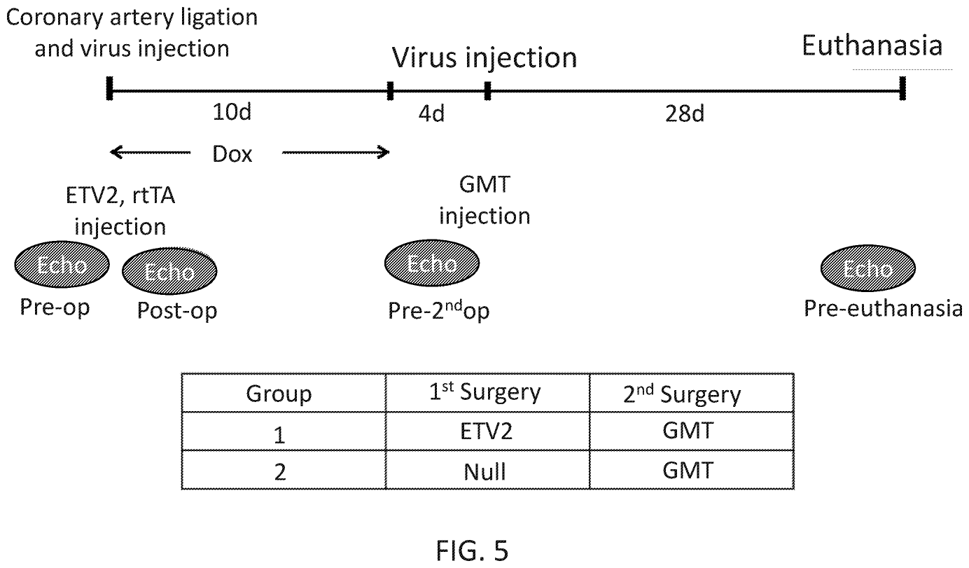

FIG. 5 illustrates an experimental design for one embodiment of an in vivo study.

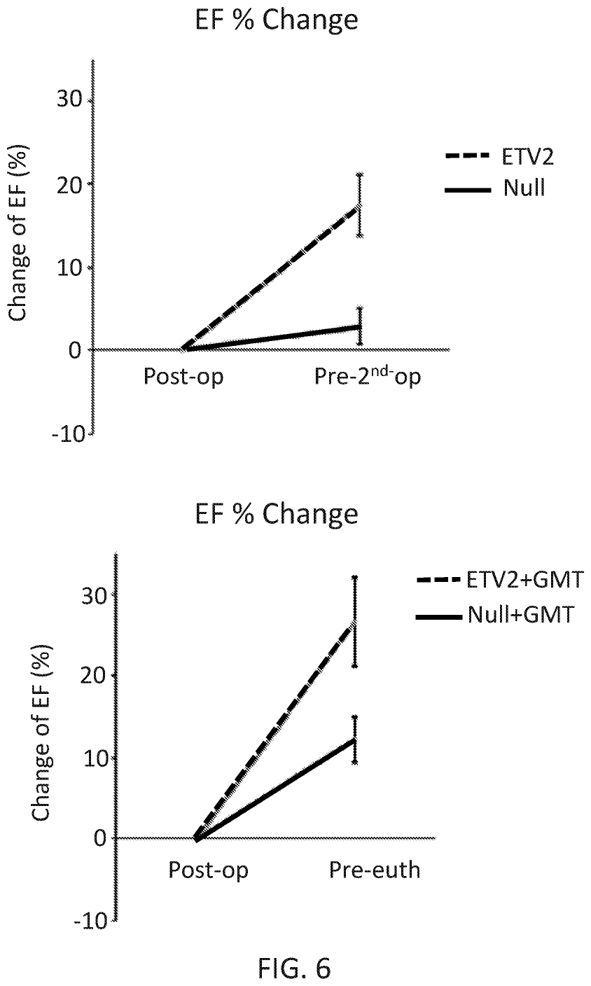

FIG. 6 shows results of echocardiography assessment for the in vivo study. The change in ejection fraction (EF) from baseline was calculated as [(EF at day 14 after the second surgery)−(EF at day 3 after the first surgery)]/(EF at day 3 after the first surgery) or ([EF at day 28 after the second surgery)−(EF at day 3 after the first surgery)]/(EF at day 3 after the first surgery). Echocardiography assessment demonstrated that ETV2 alone increased ejection fraction in the period between post-1st surgery and pre-2nd surgery (17.4±8.1 vs 2.9±4.9, p<0.01) (graph on the left side), and ejection fraction of ETV2+GMT was greater compared to GMT alone between post-1st surgery and pre-euthanasia (26.6±12.3 vs 12.2±6.1, p<0.05) (graph on the right side). Briefly, the left ventricular (LV) end-systolic and end-diastolic diameters and anterior and posterior wall thickness were measured from M-mode tracings acquired at the level of the papillary muscle. Each animal received echocardiographyic assessments 4 times, pre-first surgery, day 3 after the first surgery, pre-second surgery, and day 28 after the second surgery (see FIG. 6). The change in ejection fraction (EF) from baseline was calculated as [(EF at day 28 after the second surgery)−(EF at day 3 after the first surgery)]/(EF at day 3 after the first surgery). Echocardiography assessment demonstrated that ETV2 alone increased ejection fraction in the period between post-1st surgery and pre-2nd surgery (17.4±8.1 vs 2.9±4.9, p<0.01) (graph on the left side), and ejection fraction of ETV2+GMT was greater compared to GMT alone between post-1st surgery and pre-euthanasia (26.6±12.3 vs 12.2±6.1, p<0.05) (graph on the right side).

These data demonstrate that ETV2 administration prior to GMT administration significantly improves the efficiency of cardiac reprogramming. The data indicates that ETV2 transdifferentiation of cardiac fibroblasts into endothelial progenitors improves the differentiation efficiency of these cells into cardiomyocytes by GMT.

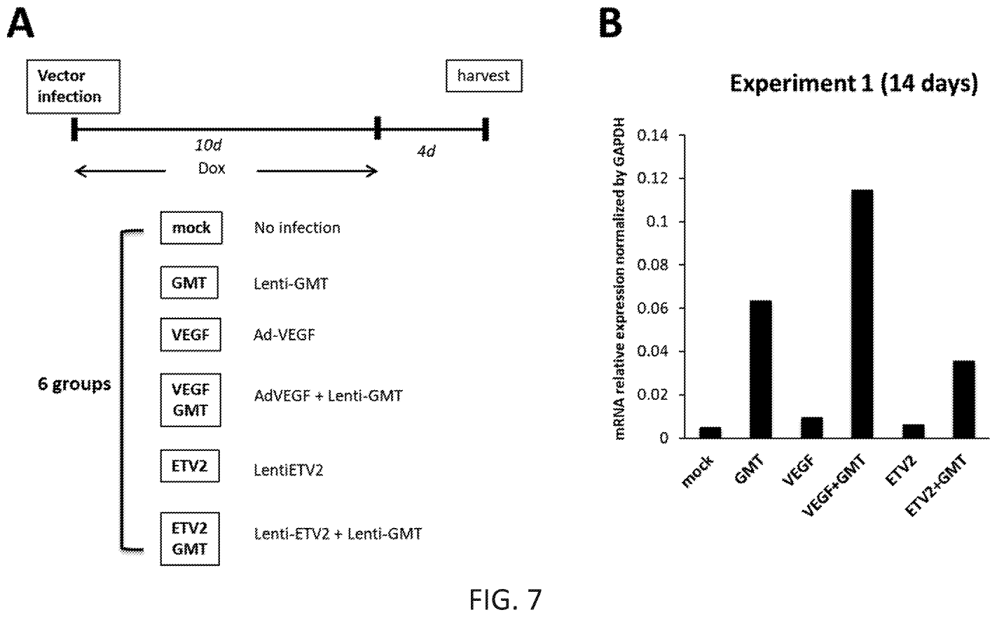

FIG. 7 shows (A) a schematic of in vitro testing protocol for simultaneous treatment of cardiac fibroblasts with VEGF or ETV2 and Gata4, Mef2c and Tbx % (GMT). “Dox” indicates doxycycline-mediated activation of ETV2. (B) Results for treatments depicted in (A), using qPCR analysis for the cardiomyocyte marker cTnT, demonstrating that simultaneous VEGF+GMT treatment of cells is superior to simultaneous ETV2+GMT treatment, and that pre-treatment of cells with VEGF yielded similar subsequent cardio-differentiation efficiency as induced by ETV2 pre-treatment.

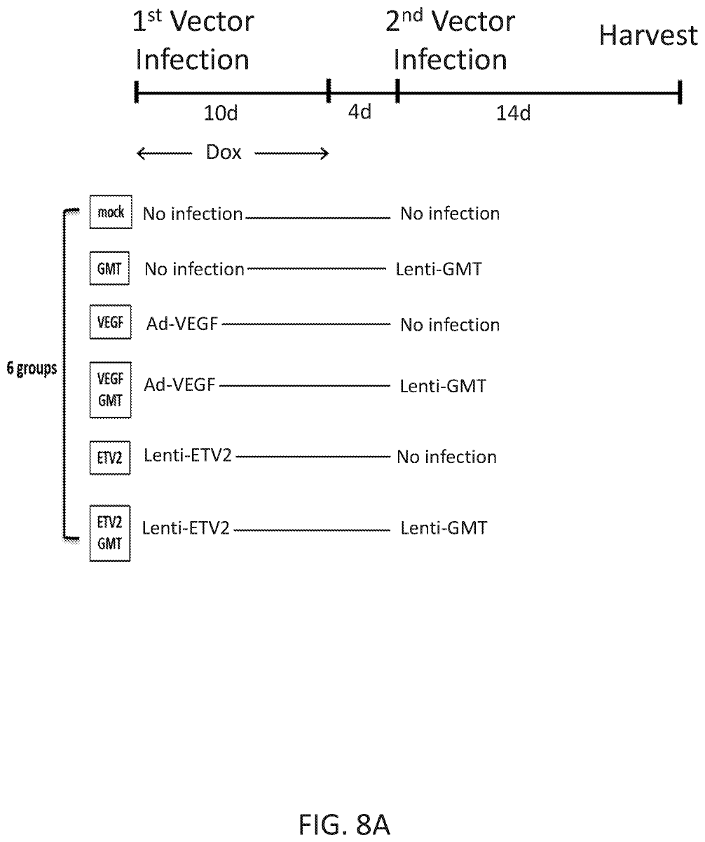

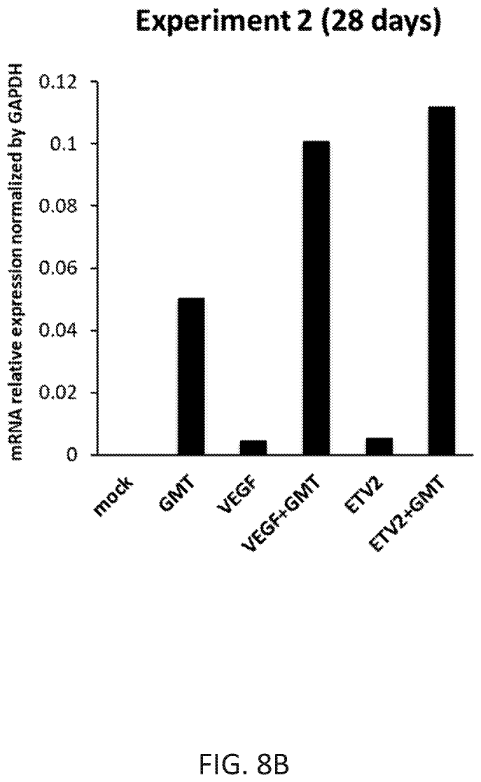

FIG. 8 shows (A) a schematic of in vitro testing protocol for sequential treatment of cardiac fibroblasts with VEGF or ETV2 and Gata4, Mef2c and Tbx % (GMT). “Dox” indicates doxycycline-mediated activation of ETV2. (B) Results for treatments depicted in (A), using qPCR analysis for the cardiomyocyte marker cTnT, demonstrating that sequential VEGF+GMT treatment of cells is superior to GMT treatment alone, and that pre-treatment of cells with VEGF yielded similar subsequent cardio-differentiation efficiency as induced by ETV2 pre-treatment.

These in vitro data confirm that these VEGF effects are independent of any promotion of angiogenesis by VEGF in models where cardiac fibroblasts are pre-treated with VEGF prior to treatment with a transdifferentiation factor. These data also confirm the previously undisclosed role of VEGF and ETV2 in inducing fibroblast to endothelial cell transdifferentiation as a means to enhance cardio-differentiation.

This novel strategy markedly improves current myocardial reprogramming strategies.

### Example 2

### Direct Cardiac Reprogramming Via Fibroblast-Endothelial Transition

### Examples of Materials and Methods

The methods disclosed herein can be applied to transfection of ETV2 and/or VEGF.

Cell culture. Commercially procured cardiac microvascular endothelial cells (AS One International Inc., Santa Clara, Calif.) were cultured on fibronectin-coated dishes in ECM-2 medium supplemented with 10 ng/ml VEGF and bFGF. For fibroblast transduction studies, adult rat cardiac tissues were harvested from 6- to 8-week-old Sprague-Dawley rats (Envigo International Holding Inc., Hackensack, N.J.) using standard cell isolation protocols. Following mincing of the tissues, cardiac fibroblasts were isolated by an explanting method in which fibroblasts migrate from minced tissue and grow in fibroblast growth medium, DMEM, 10% FBS, and 1% penicillin; streptomycin. These isolated cardiac fibroblasts were seeded on fibronectin-coated dishes in ECM-2 medium supplemented with 10 ng/ml VEGF and bFGF. For cardio-differentiation, both endothelial cells and fibroblasts were cultured in iCM medium after transduction with GATA4, Mef2c and Tbx5 (GMT).

Vectors. Lentivirus vectors encoding Gata4, Mef2c, and Tbx5 or green fluorescent protein (LentiGFP) were prepared in Gene Vector Core at BCM as previously described, as were lentivirus vectors encoding the rtTA and ETV2. rtTA (reverse tetracycline-controlled transactivator) and ETV2 plasmids were gifts from Dr. Morita. A polycicstronic-MGT plasmid was a gift from Dr. Li Qian. Retro-MGT vector was created by the Gene Vector Core as well.

Cardiac fibroblasts were infected by ETV2 and rtTA, and Doxycyclin (100 ng/ml) was added into the medium. For subsequent GMT infection, Doxycycline was stopped at day 10 because a few reports indicated that ETV2 inhibited cardiac progenitor cells to differentiate myocardial progenitor cells. Three days after the doxycycline is removed, the cells were infected by GMT.

Fluorescence-activated cell sorting (FACS) analysis. For FACS analysis, adherent cells were first washed with DPBS and trypsinized with 0.25% Trypsin/EDTA. Cells were then fixed with fixation buffer (BD Biosciences, San Jose, Calif.) for 15 minutes at room temperature. Fixed cells were washed with Perm/Wash buffer (BD Biosciences) and then incubated with mouse monoclonal anti-cardiac troponin T (cTnT) antibody (Thermo Fischer Scientific) at 1:100 dilution in Perm/Wash buffer for 90 minutes at room temperature. They were then incubated with donkey anti-mouse Alexa Fluor 647 (Invitrogen, Carlsbad, Calif.) at 1:200, washed with Perm/Wash buffer again, and then analyzed for cTnT expression using a LSR Fortessa cell sorter (BD Biosciences, Franklin Lakes, N.J.) using FlowJo software (FlowJo, LLC, Ashland, Ore). For VEGF-R2 expression analysis, mouse monoclonal anti-VEGF-R2 antibody (abcam) at 1:100 dilution was used.

qRT-PCR analysis. For qRT-PCR, total RNA was extracted using TRIzol (Invitrogen) according to the vendor's protocol. RNAs were then retro-transcribed to cDNA using iScript Supermix (Bio-Rad). qPCR was performed SYBR Green PCR Master Mix (Thermo Fisher Scientific) on a ViiA 7 Real-Time PCR System (Thermo Fisher Scientific). Results were normalized by comparative CT method with glyceraldehyde 3-phosphate dehydrogenase (GAPDH).

Immunofluorescence analysis. Immunofluorescence studies were performed on cells after 4% paraformaldehyde fixation, an permeabilization with 0.5% Triton-X solution. Cells were then blocked with 10% goat serum and incubated with primary antibodies against cTnT (1:300 dilution; Thermo Fisher Scientific), a-actinin at (1:400 dilution; Sigma-Aldrich, St. Louis, Mo.) or connexin 43 (1:400 dilution; Abcam). Goat anti-mouse Alexa 568 was used as the secondary antibody (1:1000 dilution; Thermo Fisher Scientific). Images were captured at the Core Fluorescence microscope and analyzed using ImageJ.

Statistical Analysis. Statistical analysis was performed using SAS version 9.2 (SAS Institute Inc, Cary, N.C.). Data are presented as the mean±standard deviation, unless otherwise indicated. The normality of the data was first examined using a Kolmogorov-Smirnov test. If the data have normal distribution, the analysis of variance (ANOVA) test was used. If the data did not meet normality assumption, a Krusal-Wallis test was used. If ANOVA or Krusal-Wallis test was significant for more than 2-group comparison, Bonferroni correction for ANOVA or Wicoxon rank test was followed for each pair comparison.

Results

Endothelial cells are more efficiently reprogrammed into cardiomyocyte-like cells efficiency than cardiac fibroblasts. Cardiac fibroblasts and cardiac microvascular endothelial cells were infected with lentivirus encoding GFP or GMT. After 14 days of GMT treatment, cTnT expression was observed in 13%±4% of ECs compared to 3.3%±0.1% of cardiac fibroblasts by FACS (p<0.05). Expression of the cardiac genes cTnT, Actc1, Gja1, and Hand2, were likewise significantly increased in GMT-treated ECs vs cardiac fibroblasts. Immunofluorescence studies correspondingly demonstrated much greater cTnT, a-actinin, and connexin 43 expression in ECs vs cardiac fibroblasts.

ETV2 induces EC and EndMT pathway marker expression in cardiac fibroblasts. Ten days after cardiac fibroblast infection with lentivirus encoding ETV2, FACS analysis demonstrated that a particular percentage of ETV2-infected cells expressed the endothelial cell marker VEGF-R2, whereas no VEGF-R2 expression was seen in control-treated or naïve fibroblasts. qPCR analysis likewise demonstrated upregulation of the endothelial cell markers CD31, KDR, FLi1, EGR, ESM1, Gja5, and VE cadherin compared to untreated cells.

Interestingly, ETV2 treated cells also demonstrated increased expression of markers for the EndMT expression pathway. Compered to untreated cells, FACS analysis of ETV2 treated cardiac fibroblasts demonstrated a shifted toward a CDH2+/CDH1− expression profile, indicating EndMT pathway activation. Consistent with this observation, qPCR analysis demonstrated that ETV2-treated cardiac fibroblasts demonstrated increased expression of multiple cell-plasticity and EndMT markers, including Oct4, Snail, Twist1, Zeb1, and TGFb. These data suggest that ETV2 reprogrammed cardiac fibroblasts into endothelial-like cells with transitional mesenchymal property.

Cardiac fibroblasts are more efficiently reprogrammed into cardiomyocyte-like cells by ETV2 induction prior to GMT treatment. After ten days of ETV2 treatment followed three days later by 14 days of GMT treatment, qPCR analysis demonstrated an increase in cTnT expression compared to cardiac fibroblasts treated with GMT alone (p<0.05). Similar findings were obtained with FACS analysis, which demonstrated that ETV2+GMT infected cells, compared to GMT alone (p<0.05). Immunocytochemistry likewise demonstrated greater expression of cTnT, a-actinin and connexin-43 in cells infected with GMT (as demonstrated by GFP-tagging) and ETV2 than cells treated by GMT alone.

Interestingly, ETV2-treated cardiac fibroblasts also demonstrated “spontaneous” transdifferentiation (i.e., without GMT treatment) towards cardiomyocyte-like cells compared to untreated fibroblasts. Specifically, ETV2-treated cardiac fibroblasts demonstrated increased expression of cTnT, Gata4, Mef2c, Tbx5, c-kit, Nkx2-5, and Mesp1 compared to untreated cells. Taken together, these data support the premise that ETV2-treatment of fibroblasts enhance the efficiency of their reprogramming into cardiomyocyte-like cells, in specific aspects via transdifferentiation along an EndMT pathway.

### DISCUSSION

Efforts to induce the reprogramming of one fully differentiated adult stem cell into another have proliferated ever since the initial discovery by Yamanaka of the possibility of de-differentiating adult somatic cells into induced pluripotent stem (iPS) cells, and the subsequent re-differentiation of these into a wide variety of cell types. Interestingly, the vast majority of these efforts have used mesenchymal cells, and fibroblasts in particular, as their starting cell target. This strategy has increasingly become challenged by relatively low transdifferentiation efficiency particular for human cells. This resistance to reprogramming is believed to arise from greater epigenetic controls over (reprogramming) gene activation in higher versus lower order species. “Pro-plasticity” counter-strategies that could make target cells more susceptible to reprogramming may represent a useful approach to overcoming this hindrance, as opposed to the far more prevalent strategy of adding a greater number of factors to reprogramming cocktails.

As an alternative to these fibroblast-centric approaches to cell reprogramming, the inventors questioned whether the naturally-occurring cell Endothelial Mesenchymal Transition (EndMT) pathway, which normally occurs during cell-phenotypic changes in development and inflammatory response and is characterized by pro-plasticity epigenetic modulation, might be leveraged as a strategy to enhance iCM generation from cardiac fibroblasts, which are the primary constituent of myocardial scar tissue that would be the clinical target of post-infarct myocardial regeneration strategies. This premise is supported by the previously unreported demonstration that treatment of fibroblasts with ETV2, which generates cells possessing EC and EndMT, could in turn the enhanced transdifferentiation of fibroblasts into cardiomyocyte-like cells via the subsequent treatment of ETV2-treated fibroblasts with cardio-reprogramming factors such as GMT. Interestingly, the observation of cardiomyocyte marker expression in ETV2-treated fibroblasts even without GMT treatment indicates the potency of the EndMT pathway in driving cardio-differentiation.

The focus on the endothelial cell as the axis for iCM generation has likely not been previously explored for several reasons. First, endothelial cells are relatively scarce in infarcted tissue compared to fibroblasts and would thus not be a de novo reprogramming target in this circumstance, Second, excessive endothelial cell generation in a strategy designed to therefore enhance endothelial cell target number in infarcted tissue imposes the theoretical risk of hemangioma formation, as previously shown after prolonged administration of angiogenic mediators. Third, targeting of endothelial cells, which are a critical structural component of the vasculature, poses the theoretical risk of dystopic influences of the vasculature, but this risk could be overcome, if necessary, by the incorporation of fibroblast specific promoters in the ETV2/cardio-differentiation factor vectors. In this disclosure, the inventors used rtTA system to limit duration of ETV2 activity. Because it requires further virus for rtTA, it would not be ideal for clinical use. One can utilize adenovirus or AAV virus for transient virus infection, for example. Finally, while the pro-plasticity properties of the EndMT pathway are known, there has thus far no evidence that they could be leveraged to enhance iCM generation, despite innumerable studies in this arena.

Taken together, this disclosure demonstrated that endothelial cells and cardiac fibroblasts transitioned into an endothelial cell “meso” state can be transdifferentiated into iCM cells with higher efficiency than are fibroblasts not exposed to such interventions. This alternative to a traditional fibroblast-directed strategy represents an important new approach to cardiac cell reprogramming and post-infarct myocardial regeneration in clinical post-infarct therapies.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of producing cardiomyocytes in vivo or in situ in an individual, comprising the step of delivering to the individual an effective amount of ETV2 and optionally also delivering one or more transdifferentiation factors to the individual.

2. The method of claim 1, wherein the delivering is systemic or local.

3. The method of claim 2, wherein the local delivering is by injection.

4. The method of any one of claims 1-3, wherein the delivering step is to a damaged tissue and/or organ of the individual.

5. The method of any one of claims 1-4, wherein the ETV2 and the one or more transdifferentiation factors are delivered in the same composition.

6. The method of any one of claims 1-4, wherein the ETV2 and the one or more transdifferentiation factors are delivered in different compositions.

7. The method of any one of claims 1-6, wherein the ETV2 and the one or more transdifferentiation factors are delivered at the same time.

8. The method of any one of claims 1-6, wherein the ETV2 and the one or more transdifferentiation factors are delivered at different times.

9. The method of any one of claims 1-8, wherein the ETV2 is delivered prior to the delivery of the one or more transdifferentiation factors.

10. The method of any one of claims 1-9, wherein the ETV2 is delivered as a polynucleotide or a polypeptide.

11. The method of any one of claims 1-10, wherein the one or more transdifferentiation factors are delivered as a polynucleotide or a polypeptide.

12. The method of any one of claims 1-5 and 7-11, wherein the ETV2 and the one or more transdifferentiation factors are in the form of nucleic acids that are comprised on the same vector.

13. The method of any one of claims 1-12, wherein the ETV2 and the one or more transdifferentiation factors are in the form of nucleic acids that are comprised on separate vectors.

14. The method of claim 12 or 13, wherein the vector(s) is a viral vector or a non-viral vector.

15. The method of claim 14, wherein the non-viral vector is a nanoparticle, plasmid, liposome, or a combination thereof.

16. The method of claim 14, wherein the viral vector is an adenoviral, lentiviral, retroviral, or adeno-associated viral vector.

17. The method of any of claims 12-16, wherein a promoter on the vector is a cell-specific promoter.

18. The method of any of claims 12-17, wherein a promoter on the vector is a fibroblast-specific promoter.

19. The method of claim 17 or 18, wherein the promoter is constitutive.

20. The method of any one of claims 17-19, wherein the promoter is tissue-specific.

21. The method of any one of claims 12-20, wherein the vector comprises a suicide gene.

22. The method of any one of claims 12-21, wherein the vector comprises an inducible expression element or elements.

23. The method of any one of claims 1-22, further comprising the step of delivering to the individual an additional cardiac therapy.

24. The method of claim 23, wherein the additional cardiac therapy comprises drug therapy, surgery, ventricular assist device (VAD) implantation, video assisted thoracotomy (VAT) coronary bypass, percutaneous coronary intervention (PCI), or a combination thereof.

25. The method of any one of claims 1-24, wherein the one or more transdifferentiation factors comprises GATA4, Mef2c, TBX5, or a combination thereof.

26. A composition comprising one or more nucleic acid vectors, wherein at least one vector comprises ETV2 polynucleotide and wherein at least one vector comprises a polynucleotide encoding one or more transdifferentiation factors.

27. The composition of claim 26, wherein the one or more transdifferentiation factors comprises GATA4, Mef2c, TBX5, VEGF, myocardin, Hand2, myocardin, Mesoderm posterior protein 1 (Mesp1), miR-133, miR-1, Oct4, Klf4, c-myc, Sox2, Brachyury, Nkx2.5, ETS2, ESRRG, Mrtf-A, MyoD, ZFPM2, 5-azacytidine, Zebularine, miRNA-1, miRNA-133, miRNA-208, miRNA-499, or a combination thereof.

28. A method of in vivo reprogramming of cardiac cells in an individual, comprising the step of providing locally to the heart of the individual a therapeutically effective amount of
(a) ETV2; and
(b) one or more transdifferentiation factors,
wherein the one or more transdifferentiation factors are provided to the individual at the same time or after providing the ETV2 to the individual.

29. The method of claim 28, wherein the individual has had a myocardial infarction and the ETV2 and one or more transdifferentiation factors are provided at a location in the heart that was damaged by the myocardial infarction.

30. The method of claim 28 or 29, wherein the location in the heart comprises scar tissue.

31. A method of repairing a damaged heart of an individual, comprising the step of generating cardiomyocytes from endothelial cells or endothelial-like cells in the heart of the individual upon exposure of the endothelial cells or endothelial-like cells to one or more transdifferentiation factors.

32. The method of claim 31, wherein the endothelial cells or endothelial-like cells are produced from fibroblasts that have been exposed in vivo to an effective amount of ETV2.

33. A method of producing cardiomyocytes, comprising the step of exposing Ets variant 2 (ETV2)-transfected fibroblasts, (ETV2)-transfected endothelial cells, ETV2-transfected endothelial-like cells, or a combination thereof, to one or more cardiomyocyte transdifferentiation factors, thereby producing the cardiomyocytes.

34. The method of claim 33, wherein the fibroblasts are cardiac fibroblasts.

35. The method of claim 33 or 34, wherein the one or more transdifferentiation factors are transcription factors.

36. The method of claims 33-35, wherein the one or more cardiomyocyte transdifferentiation factors comprises GATA4, myocyte enhancer factor-2c (Mef2c), T-box transcription factor 5 (TBX5), or a combination thereof.

37. The method of claim 36, wherein the transdifferentiation factors further comprise VEGF, myocardin, Hand2, myocardin, Gata4, Mef2c, Tbx5, Mesoderm posterior protein 1 (Mesp1), miR-133, miR-1, Oct4, Klf4, c-myc, Sox2, Brachyury, Nkx2.5, ETS2, ESRRG, Mrtf-A, MyoD, ZFPM2, 5-azacytidine, Zebularine, miRNA-1, miRNA-133, miRNA-208, miRNA-499, or a combination thereof.

38. The method of any one of claims 33-37, wherein following the exposing step the produced cardiomyocytes are analyzed for the expression of cardiac troponin T, GATA4, Mef2c, Tbx5, c-kit, Nkx2-5, Mesp1, or a combination thereof.

39. The method of any one of claims 33-38, wherein a therapeutically effective amount of the produced cardiomyocytes are provided to an individual in need thereof.

40. The method of any one of claims 33-39, wherein the individual has a cardiac medical condition.

41. The method of any one of claims 33-40, wherein the individual has had or is having a myocardial infarction.

42. The method of any one of claims 33-41, wherein the individual has heart damage.

43. The method of any one of claims 33-42, wherein ETV2 is expressed from a viral or non-viral vector.

44. The method of claim 43, wherein the viral vector is a lentiviral vector, adenoviral vector, adeno-associated viral vector, or retroviral vector.

45. The method of claim 43 or 44, wherein the viral vector is a lentiviral vector.

46. The method of any one of claims 33-45, wherein the expression of ETV2 and/or the expression of the one or more cardiomyocyte transdifferentiation factors is under the control of one or more regulatable expression elements.

47. The method of any one of claims 33-46, wherein the expression of ETV2 and/or the expression of the one or more cardiomyocyte transdifferentiation factors is under the control of one or more inducible regulatory elements.

48. The method of claim 47, wherein the inducible regulatory element is reverse tetracycline-controlled transactivator.

49. A method of producing differentiated cells from fibroblasts for an individual, comprising the steps of:
(a) subjecting fibroblasts to an effective amount of ETV2 to produce endothelial cells or endothelial-like cells; and
(b) subjecting the endothelial cells or endothelial-like cells to an effective amount of one or more transdifferentiation factors to produce the differentiated cells.

50. The method of claim 49, wherein step (a) and step (b) occur in vivo or in vitro.

51. The method of claim 50, wherein when the method occurs in vivo, the ETV2 and the one or more transdifferentiation factors are provided to the individual at substantially the same time.

52. The method of claim 50, wherein when the method occurs in vivo, the ETV2 is provided to the individual prior to providing the one or more transdifferentiation factors to the individual.

53. The method of claim 50, wherein when the method occurs in vitro, the ETV2 and the one or more transdifferentiation factors are provided to a culture comprising fibroblasts at substantially the same time.

54. The method of claim 50, wherein when the method occurs in vitro, the ETV2 is provided to a culture comprising fibroblasts prior to providing the one or more transdifferentiation factors to the culture.

55. The method of claim 49 or 50, wherein step (a) and step (b) occur in vivo and the ETV2 and the one or more transdifferentiation factors are provided to the individual systemically, and the one or more transdifferentiation factors are selected from the group consisting of Brn2, Mty1l, miRNA-124, Ascl1, Brn2, Myt1l, Ngn2, Ascl1, Brn2, Dimethylsulphoxide, butylated hydroxy-anisole, KCl, valproic acid, forskolin, hydrocortisone, insulin, and a combination thereof.

56. The method of claim 49 or 50, wherein step (a) and step (b) occur in vivo and the ETV2 and the one or more transdifferentiation factors are provided to the individual locally to neural tissue, and the one or more transdifferentiation factors are selected from the group consisting of Brn2, Mty1l, miRNA-124, Ascl1, Brn2, Myt1l, Ngn2, Ascl1, Brn2, Dimethylsulphoxide, butylated hydroxy-anisole, KCl, valproic acid, forskolin, hydrocortisone, insulin, and a combination thereof.

57. The method of claim 49 or 50, wherein step (a) and step (b) occur in vivo and the ETV2 and the one or more transdifferentiation factors are provided to the individual systemically, and the one or more transdifferentiation factors are selected from the group consisting of Foxa2, Hnf4α, C/EBPβ, c-Myc, Hnf1α, Hnf4α, Foxa3, Dexamethasone, oncostatin M, and a combination thereof.

58. The method of claim 49 or 50, wherein step (a) and step (b) occur in vivo and the ETV2 and the one or more transdifferentiation factors are provided to the individual locally to the liver, and the one or more transdifferentiation factors are selected from the group consisting of Foxa2, Hnf4α, C/EBPβ, c-Myc, Hnf1α, Hnf4α, Foxa3, Dexamethasone, oncostatin M, and a combination thereof.

59. The method of claim 49 or 50, wherein step (a) and step (b) occur in vivo and the ETV2 and the one or more transdifferentiation factors are provided to the individual systemically, and the one or more transdifferentiation factors are selected from the group consisting of 5-azacytidine, Myod1, SB431542, Chir99021, EGF, IGF1, and a combination thereof.

60. The method of claim 49 or 50, wherein step (a) and step (b) occur in vivo and the ETV2 and the one or more transdifferentiation factors are provided to the individual locally to skeletal muscle tissue, and the one or more transdifferentiation factors are selected from the group consisting of 5-azacytidine, Myod1, SB431542, Chir99021, EGF, IGF1, and a combination thereof.

61. The method of claim 49 or 50, wherein step (a) and step (b) occur in vivo and the ETV2 and the one or more transdifferentiation factors are provided to the individual systemically, and the one or more transdifferentiation factors are selected from the group consisting of cartilage-derived morphogenetic protein 1, c-Myc, KLF4, Sox9, and a combination thereof.

62. The method of claim 49 or 50, wherein step (a) and step (b) occur in vivo and the ETV2 and the one or more transdifferentiation factors are provided to the individual locally to cartilage tissue and/or a joint, and the one or more transdifferentiation factors are selected from the group consisting of Cartilage-derived morphogenetic protein 1, c-Myc, KLF4, Sox9, and a combination thereof.

63. The method of claim 49 or 50, wherein step (a) and step (b) occur in vivo and the ETV2 and the one or more transdifferentiation factors are provided to the individual systemically, and the one or more transdifferentiation factors are selected from the group consisting of Pdx1, Ngn3, Mafa, MAPK, STAT3, and a combination thereof.

64. The method of claim 49 or 50, wherein step (a) and step (b) occur in vivo and the ETV2 and the one or more transdifferentiation factors are provided to the individual locally to the pancreas and the one or more transdifferentiation factors are selected from the group consisting of Pdx1, Ngn3, Mafa, MAPK, STAT3, and a combination thereof.

65. The method of claim 49 or 50, wherein step (a) and step (b) occur in vivo and the ETV2 and the one or more transdifferentiation factors are provided to the individual systemically, and the one or more transdifferentiation factors are selected from the group consisting of Myod1, Dexamethasone, 1-methyl-3-isobutylxanthine, PPARγ agonists, and a combination thereof.

66. The method of claim 49 or 50, wherein step (a) and step (b) occur in vivo and the ETV2 and the one or more transdifferentiation factors are provided to the individual locally to fat tissue, and the one or more transdifferentiation factors are selected from the group consisting of Myod1, Dexamethasone, 1-methyl-3-isobutylxanthine, PPARγ agonists, and a combination thereof.

67. The method of claim 49 or 50, wherein step (a) and step (b) occur in vivo and the ETV2 and the one or more transdifferentiation factors are provided to the individual systemically, and the one or more transdifferentiation factors are selected from the group consisting of Calcitriol, dexamethasone, ascorbic acid, and beta-glycerophosphate, Runx2, MKP-1, and a combination thereof.

68. The method of claim 49 or 50, wherein step (a) and step (b) occur in vivo and the ETV2 and the one or more transdifferentiation factors are provided to the individual locally to bone tissue, and one or more transdifferentiation factors are selected from the group consisting of Calcitriol, dexamethasone, ascorbic acid, and beta-glycerophosphate, Runx2, MKP-1, and a combination thereof.

69. The method of claim 49 or 50, wherein step (a) and step (b) occur in vivo and the ETV2 and the one or more transdifferentiation factors are provided to the individual systemically, and the one or more transdifferentiation factors is selected from the group consisting of VEGF, myocardin, Hand2, myocardin, Gata4, Mef2c, Tbx5, Mesoderm posterior protein 1 (Mesp1), miR-133, miR-1, Oct4, Klf4, c-myc, Sox2, Brachyury, Nkx2.5, ETS2, ESRRG, Mrtf-A, MyoD, ZFPM2, 5-azacytidine, Zebularine, miRNA-1, miRNA-133, miRNA-208, miRNA-499, and a combination thereof.

70. The method of claim 49 or 50, wherein step (a) and step (b) occur in vivo and the ETV2 and the one or more transdifferentiation factors are provided to the individual locally to the heart, and the one or more transdifferentiation factors is selected from the group consisting of VEGF, myocardin, Hand2, myocardin, Gata4, Mef2c, Tbx5, Mesoderm posterior protein 1 (Mesp1), miR-133, miR-1, Oct4, Klf4, c-myc, Sox2, Brachyury, Nkx2.5, ETS2, ESRRG, Mrtf-A, MyoD, ZFPM2, 5-azacytidine, Zebularine, miRNA-1, miRNA-133, miRNA-208, miRNA-499, and a combination thereof.

71. Cells produced by the method of any one of claims 1-25, 28-30, and 33-70.

72. A method of producing cardiomyocytes in vivo or in situ in an individual, comprising the step of delivering to the individual an effective amount of VEGF and optionally also delivering one or more transdifferentiation factors to the individual.

73. The method of claim 72, wherein the delivering is systemic or local.

74. The method of claim 73, wherein the local delivering is by injection.

75. The method of any one of claims 72-74, wherein the delivering step is to a damaged tissue and/or organ of the individual.

76. The method of any one of claims 72-75, wherein the VEGF and the one or more transdifferentiation factors are delivered in the same composition.

77. The method of any one of claims 72-75, wherein the VEGF and the one or more transdifferentiation factors are delivered in different compositions.

78. The method of any one of claims 72-77, wherein the VEGF and the one or more transdifferentiation factors are delivered at the same time.

79. The method of any one of claims 72-77, wherein the VEGF and the one or more transdifferentiation factors are delivered at different times.

80. The method of any one of claims 72-79, wherein the VEGF is delivered prior to or after the delivery of the one or more transdifferentiation factors.

81. The method of any one of claims 72-80, wherein the VEGF is delivered as a polynucleotide or a polypeptide.

82. The method of any one of claims 72-81, wherein the one or more transdifferentiation factors are delivered as a polynucleotide or a polypeptide.

83. The method of any one of claims 72-76 and 78-82, wherein the VEGF and the one or more transdifferentiation factors are in the form of nucleic acids that are comprised on the same vector.

84. The method of any one of claims 72-83, wherein the VEGF and the one or more transdifferentiation factors are in the form of nucleic acids that are comprised on separate vectors.

85. The method of claim 83 or 84, wherein the vector(s) is a viral vector or a non-viral vector.

86. The method of claim 85, wherein the non-viral vector is a nanoparticle, plasmid, liposome, or a combination thereof.

87. The method of claim 85, wherein the viral vector is an adenoviral, lentiviral, retroviral, or adeno-associated viral vector.

88. The method of any of claims 83-87, wherein a promoter on the vector is a cell-specific promoter.

89. The method of any of claims 83-88, wherein a promoter on the vector is a fibroblast-specific promoter.

90. The method of claim 88 or 89, wherein the promoter is constitutive.

91. The method of any one of claims 88-90, wherein the promoter is tissue-specific.

92. The method of any one of claims 83-91, wherein the vector comprises a suicide gene.

93. The method of any one of claims 83-92, wherein the vector comprises an inducible expression element or elements.

94. The method of any one of claims 72-93, further comprising the step of delivering to the individual an additional cardiac therapy.

95. The method of claim 94, wherein the additional cardiac therapy comprises drug therapy, surgery, ventricular assist device (VAD) implantation, video assisted thoracotomy (VAT) coronary bypass, percutaneous coronary intervention (PCI), or a combination thereof.

96. The method of any one of claims 72-95, wherein the one or more transdifferentiation factors comprises GATA4, Mef2c, TBX5, or a combination thereof.

97. A composition comprising one or more nucleic acid vectors, wherein at least one vector comprises VEGF polynucleotide and wherein at least one vector comprises a polynucleotide encoding one or more transdifferentiation factors.

98. The composition of claim 97, wherein the one or more transdifferentiation factors comprises GATA4, Mef2c, TBX5, ETV2, myocardin, Hand2, myocardin, Mesoderm posterior protein 1 (Mesp1), miR-133, miR-1, Oct4, Klf4, c-myc, Sox2, Brachyury, Nkx2.5, ETS2, ESRRG, Mrtf-A, MyoD, ZFPM2, 5-azacytidine, Zebularine, miRNA-1, miRNA-133, miRNA-208, miRNA-499, or a combination thereof.

99. A method of in vivo reprogramming of cardiac cells in an individual, comprising the step of providing locally to the heart of the individual a therapeutically effective amount of
(a) VEGF; and
(b) one or more transdifferentiation factors,
wherein the one or more transdifferentiation factors are provided to the individual at the same time or after providing the VEGF to the individual.

100. The method of claim 99, wherein the individual has had a myocardial infarction and the VEGF and one or more transdifferentiation factors are provided at a location in the heart that was damaged by the myocardial infarction.

101. The method of claim 99 or 100, wherein the location in the heart comprises scar tissue.

102. A method of repairing a damaged heart of an individual, comprising the step of generating cardiomyocytes from endothelial cells or endothelial-like cells in the heart of the individual upon exposure of the endothelial cells or endothelial-like cells to one or more transdifferentiation factors.

103. The method of claim 102, wherein the endothelial cells or endothelial-like cells are produced from fibroblasts that have been exposed in vivo to an effective amount of VEGF.

104. A method of producing cardiomyocytes, comprising the step of exposing VEGF-transfected fibroblasts, VEGF-transfected endothelial cells, VEGF-transfected endothelial-like cells, or a combination thereof, to one or more cardiomyocyte transdifferentiation factors, thereby producing the cardiomyocytes.

105. The method of claim 104, wherein the fibroblasts are cardiac fibroblasts.

106. The method of claim 104 or 105, wherein the one or more transdifferentiation factors are transcription factors.

107. The method of claims 104-106, wherein the one or more cardiomyocyte transdifferentiation factors comprises GATA4, myocyte enhancer factor-2c (Mef2c), T-box transcription factor 5 (TBX5), or a combination thereof.

108. The method of claim 107, wherein the transdifferentiation factors further comprise myocardin, Hand2, myocardin, Gata4, Mef2c, Tbx5, ETV2, Mesoderm posterior protein 1 (Mesp1), miR-133, miR-1, Oct4, Klf4, c-myc, Sox2, Brachyury, Nkx2.5, ETS2, ESRRG, Mrtf-A, MyoD, ZFPM2, 5-azacytidine, Zebularine, miRNA-1, miRNA-133, miRNA-208, miRNA-499, or a combination thereof.

109. The method of any one of claims 104-108, wherein following the exposing step the produced cardiomyocytes are analyzed for the expression of cardiac troponin T, GATA4, Mef2c, Tbx5, c-kit, Nkx2-5, Mesp1, or a combination thereof.

110. The method of any one of claims 104-109, wherein a therapeutically effective amount of the produced cardiomyocytes are provided to an individual in need thereof.

111. The method of any one of claims 104-110, wherein the individual has a cardiac medical condition.

112. The method of any one of claims 104-111, wherein the individual has had or is having a myocardial infarction.

113. The method of any one of claims 104-112, wherein the individual has heart damage.

114. The method of any one of claims 104-113, wherein VEGF is expressed from a viral or non-viral vector.

115. The method of claim 114, wherein the viral vector is a lentiviral vector, adenoviral vector, adeno-associated viral vector, or retroviral vector.

116. The method of claim 114 or 115, wherein the viral vector is a lentiviral vector.

117. The method of any one of claims 104-116, wherein the expression of VEGF and/or the expression of the one or more cardiomyocyte transdifferentiation factors is under the control of one or more regulatable expression elements.

118. The method of any one of claims 104-117, wherein the expression of VEGF and/or the expression of the one or more cardiomyocyte transdifferentiation factors is under the control of one or more inducible regulatory elements.

119. The method of claim 118, wherein the inducible regulatory element is reverse tetracycline-controlled transactivator.

120. A method of producing differentiated cells from fibroblasts for an individual, comprising the steps of:
(a) subjecting fibroblasts to an effective amount of VEGF to produce endothelial cells or endothelial-like cells; and
(b) subjecting the endothelial cells or endothelial-like cells to an effective amount of one or more transdifferentiation factors to produce the differentiated cells.

121. The method of claim 120, wherein step (a) and step (b) occur in vivo or in vitro.

122. The method of claim 121, wherein when the method occurs in vivo, the VEGF and the one or more transdifferentiation factors are provided to the individual at substantially the same time.

123. The method of claim 121, wherein when the method occurs in vivo, the VEGF is provided to the individual prior to providing the one or more transdifferentiation factors to the individual.

124. The method of claim 121, wherein when the method occurs in vitro, the VEGF and the one or more transdifferentiation factors are provided to a culture comprising fibroblasts at substantially the same time.

125. The method of claim 121, wherein when the method occurs in vitro, the VEGF is provided to a culture comprising fibroblasts prior to providing the one or more transdifferentiation factors to the culture.

126. The method of claim 120 or 121, wherein step (a) and step (b) occur in vivo and the VEGF and the one or more transdifferentiation factors are provided to the individual systemically, and the one or more transdifferentiation factors are selected from the group consisting of Brn2, Mty1l, miRNA-124, Ascl1, Brn2, Myt1l, Ngn2, Ascl1, Brn2, Dimethylsulphoxide, butylated hydroxy-anisole, KCl, valproic acid, forskolin, hydrocortisone, insulin, and a combination thereof.

127. The method of claim 120 or 121, wherein step (a) and step (b) occur in vivo and the VEGF and the one or more transdifferentiation factors are provided to the individual locally to neural tissue, and the one or more transdifferentiation factors are selected from the group consisting of Brn2, Mty1l, miRNA-124, Ascl1, Brn2, Myt1l, Ngn2, Ascl1, Brn2, Dimethylsulphoxide, butylated hydroxy-anisole, KCl, valproic acid, forskolin, hydrocortisone, insulin, and a combination thereof.

128. The method of claim 120 or 121, wherein step (a) and step (b) occur in vivo and the VEGF and the one or more transdifferentiation factors are provided to the individual systemically, and the one or more transdifferentiation factors are selected from the group consisting of Foxa2, Hnf4α, C/EBPβ, c-Myc, Hnf1α, Hnf4α, Foxa3, Dexamethasone, oncostatin M, and a combination thereof.

129. The method of claim 120 or 121, wherein step (a) and step (b) occur in vivo and the VEGF and the one or more transdifferentiation factors are provided to the individual locally to the liver, and the one or more transdifferentiation factors are selected from the group consisting of Foxa2, Hnf4α, C/EBPβ, c-Myc, Hnf1α, Hnf4α, Foxa3, Dexamethasone, oncostatin M, and a combination thereof.

130. The method of claim 120 or 121, wherein step (a) and step (b) occur in vivo and the VEGF and the one or more transdifferentiation factors are provided to the individual systemically, and the one or more transdifferentiation factors are selected from the group consisting of 5-azacytidine, Myod1, SB431542, Chir99021, EGF, IGF1, and a combination thereof.

131. The method of claim 120 or 121, wherein step (a) and step (b) occur in vivo and the VEGF and the one or more transdifferentiation factors are provided to the individual locally to skeletal muscle tissue, and the one or more transdifferentiation factors are selected from the group consisting of 5-azacytidine, Myod1, SB431542, Chir99021, EGF, IGF1, and a combination thereof.

132. The method of claim 120 or 121, wherein step (a) and step (b) occur in vivo and the VEGF and the one or more transdifferentiation factors are provided to the individual systemically, and the one or more transdifferentiation factors are selected from the group consisting of cartilage-derived morphogenetic protein 1, c-Myc, KLF4, Sox9, and a combination thereof.

133. The method of claim 120 or 121, wherein step (a) and step (b) occur in vivo and the VEGF and the one or more transdifferentiation factors are provided to the individual locally to cartilage tissue and/or a joint, and the one or more transdifferentiation factors are selected from the group consisting of Cartilage-derived morphogenetic protein 1, c-Myc, KLF4, Sox9, and a combination thereof.

134. The method of claim 120 or 121, wherein step (a) and step (b) occur in vivo and the VEGF and the one or more transdifferentiation factors are provided to the individual systemically, and the one or more transdifferentiation factors are selected from the group consisting of Pdx1, Ngn3, Mafa, MAPK, STAT3, and a combination thereof.

135. The method of claim 120 or 121, wherein step (a) and step (b) occur in vivo and the VEGF and the one or more transdifferentiation factors are provided to the individual locally to the pancreas and the one or more transdifferentiation factors are selected from the group consisting of Pdx1, Ngn3, Mafa, MAPK, STAT3, and a combination thereof.

136. The method of claim 120 or 121, wherein step (a) and step (b) occur in vivo and the VEGF and the one or more transdifferentiation factors are provided to the individual systemically, and the one or more transdifferentiation factors are selected from the group consisting of Myod1, Dexamethasone, 1-methyl-3-isobutylxanthine, PPARγ agonists, and a combination thereof.

137. The method of claim 120 or 121, wherein step (a) and step (b) occur in vivo and the VEGF and the one or more transdifferentiation factors are provided to the individual locally to fat tissue, and the one or more transdifferentiation factors are selected from the group consisting of Myod1, Dexamethasone, 1-methyl-3-isobutylxanthine, PPARγ agonists, and a combination thereof.

138. The method of claim 120 or 121, wherein step (a) and step (b) occur in vivo and the VEGF and the one or more transdifferentiation factors are provided to the individual systemically, and the one or more transdifferentiation factors are selected from the group consisting of Calcitriol, dexamethasone, ascorbic acid, and beta-glycerophosphate, Runx2, MKP-1, and a combination thereof.

139. The method of claim 120 or 121, wherein step (a) and step (b) occur in vivo and the VEGF and the one or more transdifferentiation factors are provided to the individual locally to bone tissue, and one or more transdifferentiation factors are selected from the group consisting of Calcitriol, dexamethasone, ascorbic acid, and beta-glycerophosphate, Runx2, MKP-1, and a combination thereof.

140. The method of claim 120 or 121, wherein step (a) and step (b) occur in vivo and the VEGF and the one or more transdifferentiation factors are provided to the individual systemically, and the one or more transdifferentiation factors is selected from the group consisting of myocardin, Hand2, myocardin, Gata4, Mef2c, Tbx5, ETV2, Mesoderm posterior protein 1 (Mesp1), miR-133, miR-1, Oct4, Klf4, c-myc, Sox2, Brachyury, Nkx2.5, ETS2, ESRRG, Mrtf-A, MyoD, ZFPM2, 5-azacytidine, Zebularine, miRNA-1, miRNA-133, miRNA-208, miRNA-499, and a combination thereof.

141. The method of claim 120 or 121, wherein step (a) and step (b) occur in vivo and the VEGF and the one or more transdifferentiation factors are provided to the individual locally to the heart, and the one or more transdifferentiation factors is selected from the group consisting of myocardin, Hand2, myocardin, Gata4, Mef2c, Tbx5, ETV2, Mesoderm posterior protein 1 (Mesp1), miR-133, miR-1, Oct4, Klf4, c-myc, Sox2, Brachyury, Nkx2.5, ETS2, ESRRG, Mrtf-A, MyoD, ZFPM2, 5-azacytidine, Zebularine, miRNA-1, miRNA-133, miRNA-208, miRNA-499, and a combination thereof.

142. Cells produced by the method of any one of claims 72-96, 99-101, and 104-141


SEQUENCE LISTING (10 sequences)

SEQ ID NO: 1 - DNA, 1413 bp, Homo sapiens
ttcctgttgcagataagcccagcttagcccagctgaccccagaccctctcccctcactccccccatgtcgcaggatcgagaccctgaggcagacagcccgttcaccaagccccccgccccgcccccatcaccccgtaaacttctcccagcctccgccctgccctcacccagcccgctgttccccaagcctcgctccaagcccacgccacccctgcagcagggcagccccagaggccagcacctatccccgaggctggggtcgaggctcggccccgcccctgcctctgcaacttgagcctggctgcgacccctgctctgacgtctcggaaaattcccccttgcccaggcccttgggggagggggtgcatggtatgaaatggggctgagacccccggctgggggcagaggaacccgccagagaaggagccaaattaggcttctgtttccctgatctggcactccaaggggacacgccgacagcgacagcagagacatgctggaaaggtacaagctcatccctggcaagcttcccacagctggactggggctccgcgttactgcacccagaagttccatggggggcggagcccgactctcaggctcttccgtggtccggggactggacagacatggcgtgcacagcctgggactcttggagcggcgcctcgcagaccctgggccccgcccctctcggcccgggccccatccccgccgccggctccgaaggcgccgcgggccagaactgcgtccccgtggcgggagaggccacctcgtggtcgcgcgcccaggccgccgggagcaacaccagctgggactgttctgtggggcccgacggcgatacctactggggcagtggcctgggcggggagccgcgcacggactgtaccatttcgtggggcgggcccgcgggcccggactgtaccacctcctggaacccggggctgcatgcgggtggcaccacctctttgaagcggtaccagagctcagctctcaccgtttgctccgaaccgagcccgcagtcggaccgtgccagtttggctcgatgccccaaaactaaccaccgaggtcccattcagctgtggcagttcctcctggagctgctccacgacggggcgcgtagcagctgcatccgttggactggcaacagccgcgagttccagctgtgcgaccccaaagaggtggctcggctgtggggcgagcgcaagagaaagccgggcatgaattacgagaagctgagccggggccttcgctactactatcgccgcgacatcgtgcgcaagagcggggggcgaaagtacacgtaccgcttcgggggccgcgtgcccagcctagcctatccggactgtgcgggaggcggacggggagcagagacacaataaaaattcccggtcaaacctcaaaaaaaaaaaaaaa

SEQ ID NO: 2 - DNA, 640 bp, Homo sapiens
tcgggcctccgaaaccatgaactttctgctgtcttgggtgcattggagccttgccttgctgctctacctccaccatgccaagtggtcccaggctgcacccatggcagaaggaggggggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatct

SEQ ID NO: 3 - protein, 249 aa, Homo sapiens
MACTAWDSWSGASQTLGPAPLGPGPIPAAGSEGAAGQNCVPVAGEATSWSRAQAAGSNTSWDCSVGPDGDTYWGSGLGGEPRTDCTISWGGPAGPDCTTSWNPGLHAGGTTSLKRYQSSALTVCSEPSPQSDRASLARCPKTNHRGPIQLWQFLLELLHDGARSSCIRWTGNSREFQLCDPKEVARLWGERKRKPGMNYEKLSRGLRYYYRRDIVRKSGGRKYTYRFGGRVPSLAYPDCAGGGRGAETQ

SEQ ID NO: 4 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 5 - DNA, 3322 bp, Homo sapiens
gaccccggctgcggcgaggaggaaggagccagcctagcagcttctgcgcctgtggccgcgggtgtcctggaggcctctcggtgtgacgagtgggggacccgaaggctcgtgcgccacctccaggcctggacgctgccctccgtcttctgcccccaataggtgcgccggaccttcaggccctggggtgaattcagctgctcctacatcagcttccggaaccaccaaaaattcaaattgggattttccggagtaaacaagagcctagagccctttgctcaatgctggatttaatacgtatatatttttaagcgagttggttttttcccctttgatttttgatcttcgcgacagttcctcccacgcatattatcgttgttgccgtcgttttctctccccgcgtggctccttgacctgcgagggagagagaggacaccgaagccgggagctcgcagggaccatgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgtagatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaatcttccctcttccctcctcaaattcctgcacggacctgggacttggaggatagcaaagaaggaggccctgggctcccaggggccggcctcctctgcctggtaatgactccagaacaacaactgggaagaaacttgaagtcgacaatctggttaggggaagcgggtgttggattttctcagatgcctttacacgctgatgggactggagggagcccacccttcagcacgagcacactgcatctctcctgtgagttggagacttctttcccaagatgtccttgtcccctgcgttccccactgtggcctagaccgtgggttttgcattgtgtttctagcaccgaggatctgagaacaagcggagggccgggccctgggacccctgctccagcccgaatgacggcatctgtttgccatgtacctggatgcgacgggcccctggggacaggcccttgccccatccatccgcttgaggcatggcaccgccctgcatccctaataccaaatctgactccaaaattgtggggtgtgacatacaagtgactgaacacttcctggggagctacaggggcacttaacccaccacagcacagcctcatcaaaatgcagctggcaacttctcccccaggtgccttccccctgctgccggcctttgctccttcacttccaacatctctcaaaataaaaatccctcttcccgctctgagcgattcagctctgcccgcagcttgtacatgtctctcccctggcaaaacaagagctgggtagtttagccaaacggcaccccctcgagttcactgcagacccttcgttcaccgtgtcacacatagaggggttctgagtaagaacaaaacgttctgctgctcaagccagtctggcaagcactcagcccagcctcgaggtccttctggggagagtgtaagtggacagagtcctggtcagggggcaggagtgtcccaagggctggcccacctgctgtctgtctgctcctcctagcccttggtcagatggcagccagagtccctcaggacctgcagcctcgccccggcagaagtcttttgtccaggaggcaaaaagccagagattctgcaacacgaattcgaagcaaacaaacacaacacaacagaattcctggaaagaagacgactgctaagacacggcaggggggcctggagggagcctccgactctgagctgctccgggatctgccgcgttctcctctgcacattgctgtttctgcccctgatgctggagctcaaggagactccttcctctttctcagcagagctgtagctgactgtggcattactacgcctccccacacgcccagacccctcactccaaaatcctactggctgtagcagagaatacctttgaaccaagattctgttttaatcatcatttacattgttttcttccaaaggccccctcgtataccctccctaacccacaaacctgttaacattgtcttaaggtgaaatggctggaaaatcagtatttaactaataaatttatctgtattcctctttcaaaaaa

SEQ ID NO: 6 - protein, 443 aa, Homo sapiens
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLVDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 7 - DNA, 6194 bp, Homo sapiens
aagggggcaaagcctcggtcttcatagaaaaggagaggaggcaaacgcagcccaaactggggggtttctcttcaaagccagctggtctggctttattctgcaggaatttttttacctgtcagggtttggacaacaaagccctcagcaggtgctgacgggtacaacttcctggagaagcagaaaggcactggtgccaaagaagagttgcaaactgtgaagtaacttctatgaagagatgaagtaaagaacggaaggcaaatgattgtggcagtaaagaagtgtatgtgcaggaacgaatgcaggaatttgggaactgagctgtgcaagtgctgaagaaggagatttgtttggaggaaacaggaaagagaaagaaaaggaaggaaaaaatacataatttcagggacgagagagagaagaaaaacggggactatggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggaggcattgaacaagaaagaaaacaaaggctgtgaaagccccgatcccgactcctcttatgcactcaccccacgcactgaagaaaaatacaaaaaaattaatgaagaatttgataatatgatcaagagtcataaaattcctgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaacatgatcagattattacttactagtttttttttttttcttgcagtgtgtgtgtgtgctataccttaatggggaaggggggtcgatatgcattatatgtgccgtgtgtggaaaaaaaaaaagtcaggtactctgttttgtaaaagtacttttaaattgcctcagtgatacagtataaagataaacagaaatgctgagataagcttagcacttgagttgtacaacagaacacttgtacaaaatagattttaaggctaacttcttttcactgttgtgctcctttgcaaaatgtatgttacaatagatagtgtcatgttgcaggttcaacgttatttacatgtaaatagacaaaaggaaacatttgccaaaagcggcagatctttactgaaagagagagcagctgttatgcaacatatagaaaaatgtatagatgcttggacagacccggtaatgggtggccattggtaaatgttaggaacacaccaggtcacctgacatcccaagaatgctcacaaacctgcaggcatatcattggcgtatggcactcattaaaaaggatcagagaccattaaaagaggaccatacctattaaaaaaaaatgtggagttggagggctaacatatttaattaaataaataaataaatctgggtctgcatctcttattaaataaaaatataaaaatatgtacattacattttgcttattttcatataaaaggtaagacagagtttgcaaagcatttgtggctttttgtagtttacttaagccaaaatgtgtttttttccccttgatagcttcgctaatattttaaacagtcctgtaaaaaaccaaaaaggactttttgtatagaaagcactaccctaagccatgaagaactccatgctttgctaaccaagataactgttttctctttgtagaagttttgtttttgaaatgtgtatttctaattatataaaatattaagaatcttttaaaaaaatctgtgaaattaacatgcttgtgtatagctttctaatatatataatattatggtaatagcagaagttttgttatcttaatagcgggaggggggtatatttgtgcagttgcacatttgagtaactattttctttctgttttcttttactctgcttacattttataagtttaaggtcagctgtcaaaaggataacctgtggggttagaacatatcacattgcaacaccctaaattgtttttaatacattagcaatctattgggtcaactgacatccattgtatatactagtttctttcatgctatttttattttgttttttgcatttttatcaaatgcagggcccctttctgatctcaccatttcaccatgcatcttggaattcagtaagtgcatatcctaacttgcccatattctaaatcatctggttggttttcagcctagaatttgatacgctttttagaaatatgcccagaatagaaaagctatgttggggcacatgtcctgcaaatatggccctagaaacaagtgatatggaatttacttggtgaataagttataaattcccacagaagaaaaatgtgaaagactgggtgctagacaagaaggaagcaggtaaagggatagttgctttgtcatccgtttttaattattttaactgacccttgacaatcttgtcagcaatataggactgttgaacaatcccggtgtgtcaggacccccaaatgtcacttctgcataaagcatgtatgtcatctattttttcttcaataaagagatttaatagccatttcaagaaatcccataaagaacctctctatgtccctttttttaatttaaaaaaaatgactcttgtctaatattcgtctataagggattaattttcagaccctttaataagtgagtgccataagaaagtcaatatatattgtttaaaagatatttcagtctaggaaagattttccttctcttggaatgtgaagatctgtcgattcatctccaatcatatgcattgacatacacagcaaagaagatataggcagtaatatcaacactgctatatcatgtgtaggacatttcttatccattttttctcttttacttgcatagttgctatgtgtttctcattgtaaaaggctgccgctgggtggcagaagccaagagaccttattaactaggctatatttttcttaacttgatctgaaatccacaattagaccacaatgcacctttggttgtatccataaaggatgctagcctgccttgtactaatgttttatatattaaaaaaaaaaaatctatcaaccatttcatatatatcccactactcaaggtatccatggaacatgaaagaataacatttatgcagaggaaaaacaaaaacatccctgaaaatatacacactcatacacacacacgcacaggggaataaaataagaaaatcattttcctcaccatagacttgatcccatccttacaacccatccttctaacttgatgtgtataaaatatgcaaacatttcacaaatgttctttgtcatttcaaaatactttagtatatcaatatcagtagataccagtgggtgggaaagggtcattacatgaaaatatgaagaaatagccatattagttttttaacctgcaatttgcctcagcaacaaagaaaaagtgaatttttaatgctgaagataaagtaagctaaagtaccagcagaagccttggctatttatagcagttctgacaatagttttataagaacatgaagagaacagaatcacttgaaaatggatgccagtcatctcttgttcccactactgaattcttataaagtggtggcaagatagggaagggataatctgagaatttttaaaagatgatttaatgagaagaagcacaattttgattttgatgagtcactttctgtaaacaatcttggtctatctttacccttataccttatctgtaatttaccatttattgtatttgcaaagctagtatggtttttaatcacagtaaatcctttgtattccagactttagggcagagccctgagggagtattattttacataacccgtcctagagtaacattttaggcaacattcttcattgcaagtaaaagatccataagtggcattttacacggctgcgagtattgttatatctaatcctattttaaaagatttttggtaatatgaagcttgaatactggtaacagtgatgcaatatacgcaagctgcacaacctgtatattgtatgcattgctgcgtggaggctgtttatttcaacctttttaaaaattgtgttttttagtaaaatggcttattttttcccaaaggtggaatttagcattttgtaatgatgaatataaaaatacctgtcatccccagatcatttaaaagttaactaaagtgagaatgaaaaaacaaaattccaagacactttttaaaagaatgtctgccctcacacacttttatggatttgtttttcttacatacccatcttttaacttagagatagcattttttgccctctttattttgttgtttgtttctccagagagtaaacgctttgtagttctttctttaaaaaacattttttttaaagaagaagaagccacttgaaccctcaataaaggctgttgcctaagcatggcatacttcatctgttctcatttgtgccatctgccgtgatgtcgtcacttttatggcgttaatttcctgccactacagatcttttgaagattgctggaatactggtgtctgttagaatgcttcagactacagatgtaattaaaggcttttcttaatatgttttaaccaaagatgtggagcaatccaagccacatatcttctacatcaaatttttccattttggttattttcataatctggtattgcattttgccttccctgttcatacctcaaattgattcatacctcagtttaattcagagaggtcagttaagtgacggattctgttgtggtttgaatgcagtaccagtgttctcttcgagcaaagtagacctgggtcactgtaggcataggacttggattgcttcagatggtttgctgtatcatttttcttctttttcttttcctggggacttgtttccattaaatgagagtaattaaaatcgcttgtaaatgagggcatacaagcatttgcaacaaatattcaaatagaggctcacagcggcataagctggactttgtcgccactagatgacaagatgttataactaagttaaaccacatctgtgtatctcaagggacttaattcagctgtctgtagtgaataaaagtgggaaattttcaaaagtttctcctgctggaaataaggtataatttgtattttgcagacaattcagtaaagttactggctttcttagtgaaaaaaaaaaa

SEQ ID NO: 8 - protein, 463 aa, Homo sapiens
MGRKKIQITRIMDERNRQVTFTKRKFGLMKKAYELSVLCDCEIALIIFNSTNKLFQYASTDMDKVLLKYTEYNEPHESRTNSDIVEALNKKENKGCESPDPDSSYALTPRTEEKYKKINEEFDNMIKSHKIPAVPPPNFEMPVSIPVSSHNSLVYSNPVSSLGNPNLLPLAHPSLQRNSMSPGVTHRPPSAGNTGGLMGGDLTSGAGTSAGNGYGNPRNSPGLLVSPGNLNKNMQAKSPPPMNLGMNNRKPDLRVLIPPGSKNTMPSVNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLNIKSEPVSPPRDRTTTPSRYPQHTRHEAGRSPVDSLSSCSSSYDGSDREDHRNEFHSPIGLTRPSPDERESPSVKRMRLSEGWAT

SEQ ID NO: 9 - DNA, 2441 bp, Homo sapiens
catgccttatgcaagagacctcagtcccccggaacaactcgatttccttccaatagaggtctgaggtggactcccacctcccttcgtgaagagttccctcctctcccccttcctaagaaagtcgatcttggctctatttgtgtcttatgttcatcaccctcattcctccggagaaagccgggttggtttatgtctttatttattcccggggccaagacgtccggaacctgtggctgcgcagacccggcactgataggcgaagacggagagaaatttacctcccgccgctgccccccagccaaacgtgacagcgcgcgggccggttgcgtgactcgtgacgtctccaagtcctataggtgcagcggctggtgagatagtcgctatcgcctggttgcctctttattttactggggtatgcctggtaataaacagtaatatttaatttgtcggagaccacaaaccaaccttgagctgggaggtacgtgctcttcttgacagacgttggaagaagacctggcctaaagaggtctcttttggtggtccttttcaaagtcttcacctgagccctgctctccagcgaggcgcactcctggcttttgcgctccaaagaagaggtgggatagttggagagcagaaccttgcgcgggcacaggcctgggcgcaccatggccgacgcagacgaggctttggctggcgcacacctctggagcctgacgcaaaagacctgcctgcgattcgaaccgagagcgcgctcggggcccccagcaagtccccccggtcgtccccgcagccgccttcacccagcaggcatggagggaatcaaagtgtttctccatgaaagagaactgtggctaaaattccacgaagtcacggaaatgatcataaccaaggctggaaggcggatgtttcccagttacaaagtgaaggtgacgggcattaatcccaaaacgaagtacattcttctcatggacattgtacctgcggacgatcacagatacaaattcgcagataataaatggtgtgtgacgggcaaagctgagcccgccatggctggccgcctgtacgtgcacccagactcccccgccaccggggcgcattggatgaggcagctcgtctccttccagaaactcaagctcaccaacaaccacctggacccatttgggcatattattctaaattccatgcacaaataccagcctagattacacatcgtgaaagcggatgaaaataatggatttggctcaaaaaatacagcgttctgcactcacgtctttcctgagactgcgtttatagcagtgacttcctaccagaaccacaagatcacgcaattaaagattgagaataatccctttgccaaaggatttcggggcagtgatgacatggagctgcacagaatgtcaagaatgcaaagtaaagaatatcccgtggtccccaggagcaccgtgaggcaaaaagtggcctccaaccacagtcctttcagcagcgagtctcgagctctctccacctcatccaatttggggtcccaataccagtgtgagaatggtgtttccggcccctcccaggacctcctgcctccacccaacccatacccactgccccaggagcatagccaaatttaccattgtaccaagaggaaagaggaagaatgttccaccacagaccatccctataagaagccctacatggagacatcacccagtgaagaagattccttctaccgctctagctatccacagcagcagggcctgggtgcctcctacaggacagagtcggcacagcggcaagcttgcatgtatgccagctctgcgccccccagcgagcctgtgcccagcctagaggacatcagctgcaacacgtggccaagcatgccttcctacagcagctgcaccgtcaccaccgtgcagccatggacaggctaccctaccagcacttctccgctcacttcacctcggggcccctggtccctcggctggctggcatggcaaccatggctccccacagctgggagagggaatgttcccagcaccagacctcccgtggcccaccagcctgtggtcagcagtgtggggccccaaactggcctgcagtcccctggcacccttcagccccctgagttcctctactctcatggcgtgcaaggactctatcccctcatcagtaccactctgtgcacggagttggcatggtgcagagtggagcgacaatagctaaagtgaggcctgcttcacaacagacatttcctagagaaagagagagagagaggagaaagagagagaaggagagagacagtagccaagagaaccccacagacaagatttttcatttcacccaatgttcacatctgcactcaaggtcgctggatgctgatctaatcagtagcttgaaaccacaattttaaaaatgtgactttcttgttttgtctcaaaacttaaaaaaaaaaa

SEQ ID NO: 10 - protein, 513 aa, Homo sapiens
MADADEALAGAHLWSLTQKTCLRFEPRARSGPPASPPGRPRSRLHPAGMEGIKVFLHERELWLKFHEVTEMIITKAGRRMFPSYKVKVTGINPKTKYILLMDIVPADDHRYKFADNKWCVTGKAEPAMAGRLYVHPDSPATGAHWMRQLVSFQKLKLTNNHLDPFGHIILNSMHKYQPRLHIVKADENNGFGSKNTAFCTHVFPETAFIAVTSYQNHKITQLKIENNPFAKGFRGSDDMELHRMSRMQSKEYPVVPRSTVRQKVASNHSPFSSESRALSTSSNLGSQYQCENGVSGPSQDLLPPPNPYPLPQEHSQIYHCTKRKEEECSTTDHPYKKPYMETSPSEEDSFYRSSYPQQQGLGASYRTESAQRQACMYASSAPPSEPVPSLEDISCNTWPSMPSYSSCTVTTVQPWTGYPTSTSPLTSPRGPWSLGWLAWQPWLPTAGRGNVPSTRPPVAHQPVVSSVGPQTGLQSPGTLQPPEFLYSHGVQGLYPLISTTLCTELAWCRVERQ